(19) United States
(12) Patent Application Publication
(10) Pub. No.: US 2025/0082467 A1
(43) Pub. Date: Mar. 13, 2025

(54) HEART VALVE THAT INCLUDES COATING MATERIAL

(71) Applicant: MiRus LLC, Marietta, GA (US)

(72) Inventors: Jordan Bauman, Smyrna, GA (US); Noah Roth, Marietta, GA (US); Jay Yadav, Atlanta, GA (US); S. Beckett Gookin, Marietta, GA (US); Md Tausif Salim, Decatur, GA (US); Jorge Jimenez, Baton Rouge, LA (US); Jason White, Atlanta, GA (US)

(21) Appl. No.: 18/397,023

(22) Filed: Dec. 27, 2023

Related U.S. Application Data
(60) Provisional application No. 63/537,585, filed on Sep. 11, 2023.

(51) Int. Cl. A61F 2/24; A61L 27/04; A61L 27/06; A61L 27/30; A61L 27/54

(52) U.S. Cl. CPC A61F 2/2418; A61L 27/047; A61L 27/06; A61L 27/306; A61L 27/54; A61F 2210/0076; A61F 2250/0067; A61L 2300/114; A61L 2420/04; A61L 2420/08; A61L 2430/20

(57) ABSTRACT

A prosthetic heart valve that is at least partially coated with an enhancement coating, and a method for inserting the prosthetic heart valve in a patient. The prosthetic heart valve includes an expandable frame, a leaflet structure, and optionally an inner skirt and/or an outer skirt. One or more of the components of the prosthetic heart valve can be partially or fully coated with the enhancement coating. One type of enhancement coating that can be used includes titanium oxynitride or titanium nitride oxide (TiNOx) and/or zirconium oxynitride (ZrNxOy).

### REFERENCED APPLICATIONS

The present application claims priority to U.S. Provisional Application Ser. No. 63/537,585 filed Sep. 11, 2023, which is incorporated herein by reference.

### FIELD OF DISCLOSURE

The disclosure relates generally to medical devices and medical device applications, and more particularly to a prosthetic heart valve that is at least partially formed of a biomedical material and is partially or fully coated with a biocompatible material, and more particularly to a prosthetic heart valve that is at least partially coated with an enhancement coating. The enhancement coating can be used to reduce structural valve disease.

### BACKGROUND OF DISCLOSURE

Stainless steel, cobalt-chromium alloys, TiNi alloys, and TiAlV alloys are some of the more common metal alloys used for medical devices. For prosthetic heart valves, cobalt-chromium alloys and TiNi alloys (e.g., Nitinol) are by far the most common metal alloys used for form the frame of the prosthetic heart valve. Although these metal alloys have been successful in forming a variety of medical devices, these alloys have several deficiencies.

When a medical device is inserted into a patient, it is typically desirable for the medical device to resist ionization and/or corrosion while in the patient so as to not subject the patient to metal ions and/or oxides from the metals used to form the medical device while in the patient. Excessive ion release from the medical device can potentially be adverse to the patient. Although tradition materials such as stainless steel (316L), cobalt-chromium alloys (e.g., MP35N, etc.), Nitinol, TiAlV alloys are relatively stable when inserted into patients, some degree of metal ion release occurs when the medical device is located in the patient. However, many of the common metal alloys used for implantable medical devices include chromium and/or nickel; two elements that can cause adverse reactions in some patients. For example, nickel and/or cobalt ion release from metal alloys that includes one or both of such metals can result in allergic reaction with surrounding tissue that can in turn result in the failure of the implant.

Medical devices such as Transcatheter aortic valves (TAVs) represent a significant advancement in prosthetic heart valve technology. TAVs bring the benefit of heart valve replacement to patients that would otherwise not be operated on. Transcatheter aortic valve replacement (TAVR) can be used to treat aortic valve stenosis in patients who are classified as high-risk for open heart surgical aortic valve replacement (SAVR). Non-limiting TAVs are disclosed in U.S. Pat. Nos. 5,411,522; 6,730,118; 10,729,543; 10,820,993; 10,856,970; 10,869,761; 10,952,852; 10,980,632; 10,980,633; and US 2020/0405482, all of which are incorporated fully herein by reference. The frame material used to form the TAV is typically CoCr alloy or Nitinol. The vast majority of cardiovascular implants include valves that are made at least impart by using a CoCr alloy or Nitinol materials for construction of the structural frame of the valve.

A TAV is designed to be compressed into a small diameter catheter, remotely placed within a patient's diseased aortic valve so as to take over the function of the native valve. Some TAVs are balloon-expandable, while others are self-expandable. In both cases, the TAVs are deployed within a calcified native valve that is forced permanently open and becomes the surface against which the stent is held in place by friction. TAVs can also be used to replace failing bioprosthetic or transcatheter valves, commonly known as a valve in valve procedure. Major TAVR advantages to the traditional surgical approaches include refraining cardiopulmonary bypass, aortic cross-clamping and sternotomy that significantly reduces patients' morbidity.

However, several complications are associated with current TAV devices such as leaflet failure as a result of cells about the TAV device growing onto the frame and leaflets and ultimately interfering with the proper operation of the TAV which ultimately leads to structural valve disease (e.g., device failure or structural valve disease).

In view of the current state of the art of heart valves, there is a need for an improved heart valve that a) has reduced metal ion release as compared to medical devices formed of stainless steel, cobalt-chromium alloys, TiNi alloys, or TiAlV alloys, and/or b) addresses the problems associated with structural valve disease.

### SUMMARY OF THE DISCLOSURE

The present disclosure is direct to a prosthetic heart valve that is at least partially made of a biomedical material and a biologically compatible coating, and more particularly to a prosthetic heart valve that is at least partially (e.g. 1-99.999 wt. % and all values and ranges therebetween) or fully formed of a material that is coated with an enhancement coating used to a) reduced metal ion release of the metal material from the frame of the prosthetic heart valve, b) reduce the rate of corrosion on the metal that forms the frame of the prosthetic heart valve and/or c) reduces the rate of structural valve disease (SVD) or valve structural deterioration. Prosthetic heart valve structural deterioration is due in part to the poor hemocompatibility, poor cytocompatibility, and the susceptibility of prosthetic heart valve components to excessive tissue proliferation, inflammatory response, and/or an unfavorable healing environment. In one non-limiting embodiment of the disclosure, prosthetic heart valve structural deterioration is inhibit or reduced by i) reducing neointimal hyperplasia/cell overgrowth onto one or more portions of the prosthetic heart valve after implantation in the treatment area, ii) reducing infection about the prosthetic heart valve after implantation in the treatment area, iii) reducing platelet activation about the prosthetic heart valve after implantation in the treatment area, iv) reducing thrombosis about the prosthetic heart valve after implantation in the treatment area, v) reducing restenosis about the prosthetic heart valve after implantation in the treatment area, vi) reducing the incidence of nickel exposure and/or ion release from the frame of the prosthetic heart valve that can react with cells about the prosthetic heart valve after implantation in the treatment area, vii) reducing inflammatory cell response about the prosthetic heart valve after implantation in the treatment area, and/or viii) promoting endothelial cell angiogenesis about the prosthetic heart valve after implantation in the treatment area. In another non-limiting embodiment, the enhancement coating on one or more portions of the prosthetic heart valve is formulated to provide and/or promote generation of nitric oxide near, at and/or in adjacent tissue. Nitric oxide can reduce neointimal hyperplasia, reduce tissue proliferation, reduce platelet activation, reduce thrombosis, reduce restenosis, and can promote endothelial cell angiogenesis, all of which can contribute to an improved pro-healing environment. In another non-limiting embodiment, the enhancement coating provides, promotes and/or facilitates in a) formation or generation of nitric oxide (NO), b) stimulation of endothelial cells, and/or c) a modulation of endothelial cells. In one non-limiting arrangement, there is provided a metal oxynitride layer that is deposited a portion or all of the prosthetic heart valve. For example, the metal oxynitride layer can be deposited on a portion or all of the outer surface of a) the frame of the prosthetic heart valve, b) the inner skirt of the prosthetic heart valve, c) the outer skirt of the prosthetic heart valve, and/or d) the one or more leaflets of the prosthetic heart valve. In one non-limiting specific configuration, the metal oxynitride layer is or includes titanium oxynitride and/or zirconium oxynitride. In another non-limiting specific configuration, the thickness of the metal oxynitride layer is at least 10 nanometers (e.g., 10 nanometers to 10 microns and all values and ranges therebetween). In one non-limiting specific configuration, the oxygen to nitrogen atomic ratios of the metal oxynitride layer is 1:10 to 10:1 (and all values and ranges therebetween). In another non-limiting specific configuration, the coating of metal oxynitride layer is optionally deposited onto a metallic adhesion layer in between the base substrate (e.g., frame, inner skirt, outer skirt, one or more leaflets, etc.) and the oxynitride layer, and wherein the adhesion layer optionally is or includes titanium metal and/or zirconium metal, and wherein the adhesion layer optionally has a thickness of 10 nanometers (e.g., 10 to 500 nanometers and all values and ranges therebetween). When the metal oxynitride layer is deposited on a portion or all of the outer surface of a) the frame of the prosthetic heart valve, b) the inner skirt of the prosthetic heart valve, c) the outer skirt of the prosthetic heart valve, and/or d) the one or more leaflets of the prosthetic heart valve, the metal oxynitride coating can be used to at least partially reduces the rate of structural valve disease (SVD) by i) having the outer surface of the deployed prosthetic heart valve frame that is partially or fully coated with the metal oxynitride layer to be at least partially in direct contact with the native endothelial cells of the heart valve, ii) having the outer surface of the deployed prosthetic heart valve outer skirt that is partially or fully coated with the metal oxynitride layer to be at least partially in direct contact with the native endothelial cells of the heart valve, iii) having the outer surface of the deployed prosthetic heart valve frame that is partially or fully coated with the metal oxynitride layer to be at least partially in direct contact with the native endothelial cells of the heart valve and the blood stream that flows through the heart valve, and/or iv) having the outer surface of the deployed prosthetic heart valve outer skirt that is partially or fully coated with the metal oxynitride layer to be at least partially in direct contact with the native endothelial cells of the heart valve and the blood stream that flows through the heart valve. Nitric oxide (NO) is a short-lived, gaseous, signal molecule responsible for a plurality of cellular functions throughout the human body. NO is endogenously biosynthesized from L-arginine, oxygen, and NADPH inputs via the Nitric Oxide Synthase enzyme family. In the cardiovascular system, NO acts as a potent vasodilator. NO is also involved in cellular repair during vascular damage. A primary effect of NO is binding to Soluble Guanylyl Cyclase (sGC) activating synthesis of a downstream signaling molecule Cyclic Guanosine Monophosphate (cGMP). cGMP is subsequently responsible for downregulation of factors responsible for platelet aggregation, apoptosis, inflammation, and tissue remodeling. cGMP is also responsible for upregulation of factors responsible for vasodilation. Examples of direct nitric oxide donors includes agents with nitroso or nitrosyl functional groups that spontaneously release nitric oxide. Examples of metabolic nitric oxide donors include agents with organic nitrate and nitrite esters requiring enzymatic metabolism to generate bioactive nitric oxide. Examples of bifunctional nitric oxide donors include agents with nitrate esters and S-nitrosothiols that release nitric oxide simultaneously with performing additional pharmacological benefits.

In accordance with one non-limiting aspect of the present disclosure, the prosthetic heart valve is not limited to a TAV, but can be mitral valve replacement, tricuspid valve replacement, pulmonary or valve replacement. The prosthetic heart valve includes a radially collapsible and expandable frame and a leaflet structure comprising a plurality of leaflets. The prosthetic heart valve can optionally include an annular skirt or cover member disposed on and covering the open cells on the open cell frame of at least a portion of the frame. The frame can comprise a plurality of interconnected struts and strut joints defining a plurality of open cells in the frame. The frame is partially (e.g., 1-99.999 wt. % and all values and ranges therebetween) or fully made of a metal material.

In accordance with another and/or alternative non-limiting aspect of the present disclosure, there is provided a prosthetic heart valve that includes a frame, a leaflet structure supported by the frame, and an optional inner skirt secured to the surface of the frame and/or leaflet structure. The prosthetic heart valve can be implanted in the annulus of the native aortic valve; however, the prosthetic heart valve also can be configured to be implanted in other valves of the heart (e.g., tricuspid valve, pulmonary valve, mitral valve). The prosthetic heart valve has a “lower” end and an “upper” end, wherein the lower end of the prosthetic heart valve is the inflow end and the upper end of the prosthetic heart valve is the outflow end. The metal alloy that is used to partially or fully form the frame of the prosthetic heart valve is configured to be radially collapsible to a collapsed or crimped state for introduction into the body (e.g., on a delivery catheter, etc.) and radially expandable to an expanded state for implanting the prosthetic heart valve at a desired location in the body (e.g., the aortic valve, tricuspid valve, pulmonary valve, mitral valve, etc.). The frame of the prosthetic heart valve can be formed of a plastically-expandable material that permits crimping of the frame to a smaller profile for delivery and expansion of the frame at the treatment site. The expansion of the crimped frame of the prosthetic heart valve can be by an expansion device such as, but not limited to, a balloon of on a balloon catheter; however, the frame can optionally be partially (e.g., 1-99.999 wt. % and all values and ranges therebetween) or fully formed of a self-expanding material (e.g., Nitinol, etc.). The frame can be at least partially (e.g., 1-99.999 wt. % and all values and ranges therebetween) formed of a plurality of angularly spaced, vertically extending posts, and/or struts. The posts and/or struts can optionally be interconnected via a lower row of circumferentially extending struts and an upper row of circumferentially extending struts via strut joints. The struts can be arrangement in a variety of patterns (e.g., zig-zag pattern, saw-tooth pattern, triangular pattern, polygonal pattern, oval pattern, etc.). One or more of the posts and/or struts can have the same or different thicknesses and/or cross-sectional shape and/or cross-sectional area.

In accordance with another and/or alternative non-limiting aspect of the present disclosure, there is provided a prosthetic heart valve that includes an inner skirt that can be formed of a variety of flexible materials (e.g., polymer [e.g., polyethylene terephthalate (PET), polyester, nylon, Kevlar®, silicon, etc.], composite material, metal, fabric material, etc.). In one non-limiting embodiment, the material used to partially (e.g., 1-99.999 wt. % and all values and ranges therebetween) or fully form the inner skirt can optionally be substantially non-elastic (i.e., substantially non-stretchable and non-compressible). In another non-limiting embodiment, the material used to partially or fully form the inner skirt can optionally be a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). The inner skirt can optionally be formed from a combination of a cloth or fabric material that is coated with a flexible material or with a stretchable and/or compressible material so as to provide additional structural integrity to the inner skirt. The size, configuration, and thickness of the inner skirt is non-limiting (e.g., thickness of 0.1-20 mils and all values and ranges therebetween). The inner skirt can be secured to the inside and/or outside of the frame using various means (e.g., sutures, clamp arrangement, etc.).

In accordance with another and/or alternative non-limiting aspect of the present disclosure, there is provided a prosthetic heart valve that optionally includes an inner skirt that can be used to 1) at least partially seal and/or prevent perivalvular leakage, 2) at least partially secure the leaflet structure to the frame, 3) at least partially protect the leaflets from damage during the crimping and/or expansion process, and/or 4) at least partially protect the leaflets from damage during the operation of the prosthetic heart valve in the heart.

In accordance with another and/or alternative non-limiting aspect of the present disclosure, there is provided a prosthetic heart valve that optionally includes an outer or sleeve that is positioned at least partially (e.g., 1-99.999 wt. % and all values and ranges therebetween) about the exterior region of the frame. The outer skirt or sleeve generally is positioned completely around a portion of the outside of the frame. Generally, the outer skirt is positioned about the lower portion of the frame, but does not fully cover the upper half of the frame; however, this is not required. The outer skirt can be connected to the frame by a variety of arrangements (e.g., sutures, adhesive, melted connection, clamping arrangement, etc.). At least a portion of the outer skirt can optionally be located on the interior surface of the frame. Generally, the outer skirt is formed of a more flexible and/or compressible material than the inner skirt; however, this is not required. The outer skirt can be formed of a variety of a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). The outer skirt can optionally be formed from a combination of a cloth or fabric material that is coated with the stretchable and/or compressible material to provide additional structural integrity to the outer skirt. The size, configuration, and thickness of the outer skirt is non-limiting. The thickness of the outer skirt is generally at least 0.1 mils (e.g., 0.1-20 mils and all values and ranges therebetween).

In accordance with another and/or alternative non-limiting aspect of the present disclosure, there is provided a prosthetic heart valve that includes a leaflet structure that can be can be attached to the frame and/or skirt. The connection arrangement used to secure the leaflet structures to the frame and/or skirt is non-limiting (e.g., sutures, staples, melted bold, adhesive, clamp arrangement, etc.). The material used to form the leaflet structures include bovine pericardial tissue, biocompatible synthetic materials, or various other suitable natural or synthetic materials.

In accordance with another and/or alternative non-limiting aspect of the present disclosure, there is provided a prosthetic heart valve that includes a leaflet structure comprised of two or more leaflets (e.g., 2, 3, 4, 5, 6, etc.). In one non-limiting arrangement, the leaflet structure includes three leaflets arranged to collapse in a tricuspid arrangement. The configuration of the leaflet structures is non-limiting.

In accordance with another and/or alternative non-limiting aspect of the present disclosure, there is provided a prosthetic heart valve that includes a leaflet structure wherein the leaflets of the leaflet structure can optionally be secured to one another at their adjacent sides to form commissures of the leaflet structure (the edges where the leaflets come together). The leaflet structure can be secured together by a variety of connection arrangement (e.g., sutures, adhesive, melted bond, clamping arrangement, etc.).

In accordance with another and/or alternative non-limiting aspect of the present disclosure, there is provided a prosthetic heart valve that includes a leaflet structure wherein one or more of the leaflets can optionally include reinforcing structures or strips to 1) facilitate in securing the leaflets together, 2) facilitate in securing the leaflets to the skirt and/or frame, and/or 3) inhibit or prevent tearing or other types of damage to the leaflets.

In accordance with another and/or alternative non-limiting aspect of the present disclosure, there is provided a method for crimping a prosthetic heart valve having a frame. The method includes placing the prosthetic heart valve in the crimping aperture of a crimping device such that the frame of the prosthetic heart valve is disposed adjacent to the crimping jaws of the crimping device. Pressure is applied against the frame with the crimping jaws to radially crimp the prosthetic heart valve to a smaller profile.

In accordance with another and/or alternative non-limiting aspect of the present disclosure, the frame of the prosthetic heart valve is partially (e.g. 1-99.999 wt. % and all values and ranges therebetween) or fully formed of a metal material that includes a) standard stainless steel, b) standard CoCr alloy or standard MP35N alloy or a standard Phynox alloy or standard Elgiloy alloy or standard L605 alloy, c) standard TiAlV alloy, d) standard aluminum alloy, c) standard nickel alloy, f) standard titanium alloy, g) standard tungsten alloy, h) standard molybdenum alloy, i) standard copper alloy, j) standard beryllium-copper alloy, k) standard Nitinol alloy, l) refractory metal alloy, or m) metal alloy that includes at least 5 atomic weight percent (awt. %) or atomic percent (awt %) rhenium (e.g., 5-99 awt. % rhenium and all values and ranges therebetween). As used herein, atomic weight percent (awt. %) or atomic percent (awt %) are used interchangeably. As defined herein, the weight percentage (wt. %) of an element is the weight of that element measured in the sample divided by the weight of all elements in the sample multiplied by 100. The atomic percentage or atomic weight percent (awt %) is the number of atoms of that element, at that weight percentage, divided by the total number of atoms in the sample multiplied by 100. The use of the terms weight percentage (wt. %) and atomic percentage or atomic weight percentage (awt. %) are two ways of referring to metallic alloy and its constituents. As defined herein, a standard stainless-steel alloy (SS alloy) includes 10-28 wt. % (weight percent) chromium, 0-35 wt. % nickel, 0-4 wt. % molybdenum, 0-2 wt. % manganese, 0-0.75 wt. % silicon, 0-0.3 wt. % carbon, 0-5 wt. % titanium, 0-10 wt. % niobium, 0-5 wt. % copper, 0-4 wt. % aluminum, 0-10 wt. % tantalum, 0-1 wt. % Se, 0-2 wt. % vanadium, 0-2 wt. % tungsten, and at least 50 wt. % iron. A standard 316L alloy that falls within a standard stainless-steel alloy includes 17-19 wt. % chromium, 13-15 wt. % nickel, 2-4 wt. % molybdenum, 2 wt. % max manganese, 0.75 wt. % max silicon, 0.03 wt. % max carbon, balance iron. As defined herein, a standard cobalt-chromium alloy (CoCr alloy) includes 15-32 wt. % chromium, 1-38 wt. % nickel, 2-18 wt. % molybdenum, 0-18 wt. % iron, 0-1 wt. % titanium, 0-0.15 wt. % manganese, 0-0.15 wt. % silver, 0-0.25 wt. % carbon, 0-16 wt. % tungsten, 0-2 wt. % silicon, 0-2 wt. % aluminum, 0-1 wt. % iron, 30-68 wt. % cobalt, 0-0.1 wt. % boron, 0-0.15 wt. % silver, and 0-2 wt. % titanium. As a standard MP35N alloy that falls within a standard CoCr alloy includes 18-22 wt. % chromium, 32-38 wt. % nickel, 8-12 wt. % molybdenum, 0-2 wt. % iron, 0-0.5 wt. % silicon, 0-0.5 wt. % manganese, 0-0.2 wt. % carbon, 0-2 wt. % titanium, 0-0.1 wt. %, 0-0.1 wt. % boron, 0-0.15 wt. % silver, and balance cobalt. As defined herein, a standard Phynox and standard Elgiloy alloy that falls within a standard CoCr alloy includes 38-42 wt. % cobalt, 18-22 wt. % chromium, 14-18 wt. % iron, 13-17 wt. % nickel, 6-8 wt. % molybdenum. As defined herein, a standard L605 alloy that falls within a standard CoCr alloy includes 18-22 wt. % chromium, 14-16 wt. % tungsten, 9-11 wt. % nickel, balance cobalt. As defined herein, a standard titanium-aluminum-vanadium alloy (TiAlV alloy) includes 5.5-6.75 wt. % aluminum, 3.5-4.5 wt. % vanadium, 85-93 wt. % titanium, 0-0.4 wt. % iron, 0-0.2 wt. % carbon. A standard Ti-6Al-4V alloy that falls with a standard TiAlV alloy includes incudes 3.5-4.5 wt. % vanadium, 5.5-6.75 wt. % aluminum, 0.3 wt. % max iron, 0.08 wt. % max carbon, 0.05 wt. % max yttrium, balance titanium. As defined herein, a standard aluminum alloy includes 80-99 wt. % aluminum, 0-12 wt. % silicon, 0-5 wt. % magnesium, 0-1 wt. % manganese, 0-0.5 wt. % scandium, 0-0.5 wt. % beryllium, 0-0.5 wt. % yttrium, 0-0.5 wt. % cerium, 0-0.5 wt. % chromium, 0-3 wt. % iron, 0-0.5, 0-9 wt. % zinc, 0-0.5 wt. % titanium, 0-3 wt. % lithium, 0-0.5 wt. % silver, 0-0.5 wt. % calcium, 0-0.5 wt. % zirconium, 0-1 wt. % lead, 0-0.5 wt. % cadmium, 0-0.05 wt. % bismuth, 0-1 wt. % nickel, 0-0.2 wt. % vanadium, 0-0.1 wt. % gallium, and 0-7 wt. % copper. As defined herein, a standard nickel alloy includes 30-98 wt. % nickel, 5-25 wt. % chromium, 0-65 wt. % iron, 0-30 wt. % molybdenum, 0-32 wt. % copper, 0-32 wt. % cobalt, 2-2 wt. % aluminum, 0-6 wt. % tantalum, 0-15 wt. % tungsten, 0-5 wt. % titanium, 0-6 wt. % niobium, 0-3 wt. % silicon. As defined herein, a standard titanium alloy includes 80-99 wt. % titanium, 0-6 wt. % aluminum, 0-3 wt. % tin, 0-1 wt. % palladium, 0-8 wt. % vanadium, 0-15 wt. % molybdenum, 0-1 wt. % nickel, 0-0.3 wt. % ruthenium, 0-6 wt. % chromium, 0-4 wt. % zirconium, 0-4 wt. % niobium, 0-1 wt. % silicon, 0.0.5 wt. % cobalt, 0-2 wt. % iron. As defined herein, a standard tungsten alloy includes 85-98 wt. % tungsten, 0-8 wt. % nickel, 0-5 wt. % copper, 0-5 wt. % molybdenum, 0-4 wt. % iron. As defined herein, a standard molybdenum alloy includes 90-99.5 wt. % molybdenum, 0-1 wt. % nickel, 0-1 wt. % titanium, 0-1 wt. % zirconium, 0-30 wt. % tungsten, 0-2 wt. % hafnium, 0-2 wt. % lanthanum. As defined herein, a standard copper alloy includes 55-95 wt. % copper, 0-40 wt. % zinc, 0-10 wt. % tin, 0-10 wt. % lead, 0-1 wt. % iron, 0-5 wt. % silicon, 0-12 wt. % manganese, 0-12 wt. % aluminum, 0-3 wt. % beryllium, 0-1 wt. % cobalt, 0-20 wt. % nickel. As defined herein, a standard beryllium-copper alloy includes 95-98.5 wt. % copper, 1-4 wt. % beryllium, 0-1 wt. % cobalt, and 0-0.5 wt. % silicon. As defined herein, a standard titanium-nickel alloy (e.g., Nitinol alloy) includes 42-58 wt. % nickel and 42-58 wt. % titanium. As defined herein, a refractory metal alloy is a metal alloy that includes at least 20 wt. % of one or more of molybdenum, rhenium, niobium, tantalum or tungsten. Non-limiting refractory metal alloys include MoRe alloy, ReW alloy, MoReCr alloy, MoReTa alloy, MoReTi alloy, WCu alloy, ReCr, molybdenum alloy, rhenium alloy, tungsten alloy, tantalum alloy, niobium alloy, etc.

In accordance with another and/or alternative non-limiting aspect of the present disclosure, the frame of the prosthetic heart valve is partially or fully formed of a metal material that includes a metal alloy that contains at least 15 awt. % rhenium. It has been found that for several metal alloys the inclusion of at least 15 awt % rhenium results in the ductility and/or tensile strength of the metal alloy to improve as compared to a metal alloy is that absent rhenium. Such improvement in ductility and/or tensile strength due to the inclusion of at least 15 awt. % rhenium in the metal alloy is referred to as the “rhenium effect.” As defined herein, a “rhenium effect” is a) an increase of at least 10% in ductility of the metal alloy caused by the addition of rhenium to the metal alloy, and/or b) an increase of at least 10% in tensile strength of the metal alloy caused by the addition of rhenium to the metal alloy. It has been found for some metal alloys (e.g., standard stainless steel, standard CoCr alloys, standard TiAlV alloys, standard aluminum alloys, standard nickel alloys, standard titanium alloys, standard tungsten alloys, standard molybdenum alloys, standard copper alloys, standard MP35N alloys, standard beryllium-copper alloys, etc.), the inclusion of at least 15 awt. % rhenium results in improved ductility and/or tensile strength. It has been found that the addition of rhenium to a metal alloy can result in the formation of a twining alloy in the metal alloy that results in the overall ductility of the metal alloy to increase as the yield and tensile strength increases as a result of reduction and/or work hardening of the metal alloy that includes the rhenium addition. The rhenium effect has been found to occur when the atomic weight of rhenium in the metal alloy is at least 15% (e.g., 15-99 awt. % rhenium in the metal alloy and all values and ranges therebetween). For example, for standard stainless-steel alloys, the rhenium effect can begin to be present when the stainless-steel alloy is modified to include a rhenium amount of at least 5-10 wt. % (and all values and ranges therebetween) of the stainless-steel alloy. For standard CoCr alloys, the rhenium effect can begin to be present when the CoCr alloy is modified to include a rhenium amount of at least 4.8-9.5 wt. % (and all values and ranges therebetween) of the CoCr alloy. For standard TiAlV alloys, the rhenium effect can begin to be present when the TiAlV alloy is modified to include a rhenium amount of at least 4.5-9 wt. % (and all values and ranges therebetween) of the TiAlV alloy. It can be appreciated, the rhenium content in the above non-limiting examples can be greater than the minimum amount to create the rhenium effect in the metal alloy.

In accordance with another and/or alternative aspect of the present disclosure, the metal alloy that is used to partially or fully form the frame of the prosthetic heart valve includes at least 5 awt. % (e.g., 5-99 awt. % and all values and ranges therebetween) rhenium, and 0.1-96 wt. % (and all values and ranges therebetween) of one or more additives selected from the group of aluminum, boron, beryllium, bismuth, cadmium, calcium, cerium, chromium, cobalt, copper, gallium, gold, hafnium, iridium, iron, lanthanum, lanthanum oxide, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and the metal alloy optionally includes 0-2 wt. % (and all values and ranges therebetween) of a combination of other metals (e.g., metals other than additives), carbon, oxygen, phosphorous, sulfur, hydrogen and/or nitrogen, and which metal alloy exhibits a rhenium effect. In one non-limiting embodiment, the metal alloy that is used to partially or fully form the frame of the prosthetic heart valve is a standard stainless-steel alloy that has been modified to include at least 15 awt. % rhenium. In another non-limiting embodiment, the metal alloy that is used to partially or fully form the frame of the prosthetic heart valve is a standard cobalt chromium alloy that has been modified to include at least 15 awt. % rhenium. In another non-limiting embodiment, the metal alloy that is used to partially or fully form the frame of the prosthetic heart valve is a standard TiAlV alloy that has been modified to include at least 15 awt. % rhenium. In another non-limiting embodiment, the metal alloy that is used to partially or fully form the frame of the prosthetic heart valve is a standard aluminum alloy that has been modified to include at least 15 awt. % rhenium. In another non-limiting embodiment, the metal alloy that is used to partially or fully form the frame of the prosthetic heart valve is a standard nickel alloy that has been modified to include at least 15 awt. % rhenium. In another non-limiting embodiment, the metal alloy that is used to partially or fully form the frame of the prosthetic heart valve is a standard titanium alloy that has been modified to include at least 15 awt. % rhenium. In another non-limiting embodiment, the metal alloy that is used to partially or fully form the frame of the prosthetic heart valve is a standard tungsten alloy that has been modified to include at least 15 awt. % rhenium. In another non-limiting embodiment, the metal alloy that is used to partially or fully form the frame of the prosthetic heart valve is a standard molybdenum alloy that has been modified to include at least 15 awt. % rhenium. In another non-limiting embodiment, the metal alloy that is used to partially or fully form the frame of the prosthetic heart valve is a standard copper alloy that has been modified to include at least 15 awt. % rhenium. In another non-limiting embodiment, the metal alloy that is used to partially or fully form the frame of the prosthetic heart valve is a standard beryllium-copper alloy that has been modified to include at least 15 awt. % rhenium.

In accordance with another and/or alternative aspect of the present disclosure, the metal alloy that is used to partially or fully form the frame of the prosthetic heart valve includes rhenium and molybdenum, and the weight percent of rhenium in the metal alloy is optionally greater than the weight percent of molybdenum in the metal alloy, and the weight percent of one or more additive (e.g., aluminum, boron, beryllium, bismuth, cadmium, calcium, cerium, chromium, cobalt, copper, gallium, gold, hafnium, iridium, iron, lanthanum, lanthanum oxide, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium) in the metal alloy is optionally greater that the weight percent of molybdenum in the metal alloy, and the metal alloy optionally includes 0-2 wt. % of a combination of other metals (metals other than the additive), carbon, oxygen, phosphorous, sulfur, hydrogen and/or nitrogen. In one non-limiting embodiment, the metal alloy that is used to partially or fully form the frame of the prosthetic heart valve includes rhenium and molybdenum, and the weight percent of rhenium plus the combined weight percent of additives is greater than the weight percent of molybdenum, and the metal alloy optionally includes 0-2 wt. % of a combination of other metals (metals other than the additive), carbon, oxygen, phosphorous, sulfur, hydrogen and/or nitrogen.

In accordance with another and/or alternative aspect of the present disclosure, the metal alloy that is used to partially or fully form the frame of the prosthetic heart valve includes rhenium and molybdenum, and the atomic weight percent of rhenium to the atomic weight percent of the combination of one or more of bismuth, niobium, tantalum, tungsten, titanium, vanadium, chromium, manganese, yttrium, zirconium, technetium, ruthenium, rhodium, hafnium, osmium, copper, and iridium is 0.4:1 to 2.5:1 (and all values and ranges therebetween).

In accordance with another and/or alternative aspect of the present disclosure, the metal alloy that is used to partially or fully form the frame of the prosthetic heart valve includes at least 5 awt. % (e.g., 5-99 awt. % and all values and ranges therebetween) rhenium plus at least two metals selected from the group of molybdenum, bismuth, chromium, iridium, niobium, tantalum, titanium, yttrium, and zirconium, and the content of the metal alloy that includes other elements and compounds is 0-0.1 wt. %. In another non-limiting embodiment, the metal alloy includes rhenium, molybdenum, and chromium. In another non-limiting embodiment, the metal alloy includes at least 35 wt. % (e.g., 35-75 wt. % and all values and ranges therebetween) rhenium, and the metal alloy also includes chromium. In one non-limiting embodiment, the metal alloy includes at least 35 wt. % rhenium and at least 25 wt. % (e.g., 25-49.9 wt. % and all values and ranges therebetween) of the metal alloy includes chromium, and optionally 0.1-40 wt. % (and all values and ranges therebetween) of the metal alloy includes one or more of aluminum, boron, beryllium, bismuth, cadmium, calcium, cerium, chromium, cobalt, copper, gallium, gold, hafnium, iridium, iron, lanthanum, lanthanum oxide, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and the metal alloy optionally includes 0-2 wt. % (and all values and ranges therebetween) of a combination of other metals, carbon, oxygen, phosphorous, sulfur, hydrogen and/or nitrogen. In another non-limiting embodiment, the metal alloy includes 15-50 awt. % rhenium (and all values and ranges therebetween) and 0.5-70 awt. % chromium (and all values and ranges therebetween). In another non-limiting embodiment, the metal alloy includes 15-50 awt. % rhenium (and all values and ranges therebetween) and 0.5-70 awt. % tantalum (and all values and ranges therebetween). In another non-limiting embodiment, the metal alloy includes 15-50 awt. % rhenium (and all values and ranges therebetween) and 0.5-70 awt. % niobium (and all values and ranges therebetween). In another non-limiting embodiment, the metal alloy includes 15-50 awt. % rhenium (and all values and ranges therebetween) and 0.5-70 awt. % titanium (and all values and ranges therebetween). In another non-limiting embodiment, the metal alloy includes 15-50 awt. % rhenium (and all values and ranges therebetween) and 0.5-70 awt. % zirconium (and all values and ranges therebetween). In another non-limiting embodiment, the metal alloy includes 15-50 awt. % rhenium (and all values and ranges therebetween) and 0.5-70 awt. % molybdenum (and all values and ranges therebetween). In another non-limiting embodiment, the metal alloy includes at least 15 awt. % rhenium, greater than 50 wt. % titanium (e.g., 51-80 wt. % and all values and ranges therebetween), 15-45 wt. % (and all values and ranges therebetween) niobium, 0-10 wt. % (and all values and ranges therebetween) zirconium, 0-15 wt. % (and all values and ranges therebetween) tantalum, and 0-8 wt. % molybdenum (and all values and ranges therebetween).

Several non-limiting examples of metal alloys that can be used to partially or fully form the orthopedic medical device are set forth below in weight percent:

| | Component/Wt. % | | | |
|---|---|---|---|---|
| | Ex. 1 | Ex. 2 | Ex. 3 | Ex. 4 |
| Al | 0-35% | 0-30% | 0-25% | 0-10% |
| Bi | 0-20% | 0-20% | 0-20% | 0-20% |
| Cr | 0-60% | 0-35% | 0-30% | 0-25% |
| Co | 0-60% | 0-50% | 0-40% | 0-20% |
| Mo | 0-95% | 0-80% | 0-55% | 0-30% |
| Nb | 0-80% | 0-60% | 0-50% | 0-20% |
| Ni | 0-60% | 0-55% | 0-40% | 0-20% |
| Re | 0.1-70% | 4.5-70% | 5-70% | 5-70% |
| Ta | 0-80% | 0-50% | 0-40% | 0-25% |
| Ti | 0-60% | 0-55% | 0-40% | 0-20% |
| V | 0-20% | 0-15% | 0-10% | 0-10% |
| W | 0-80% | 0-70% | 0-50% | 0-20% |
| Y | 0-20% | 0-15% | 0-10% | 0-10% |
| Zr | 0-20% | 0-15% | 0-10% | 0-10% |

| | Component/Wt. % | | | |
|---|---|---|---|---|
| | Ex. 5 | Ex. 6 | Ex. 7 | Ex. 8 |
| Ag | 0-20% | 0-20% | 0-20% | 0-20% |
| Al | 0-35% | 0-30% | 5-30% | 0-25% |
| Bi | 0-20% | 0-20% | 0-20% | 0-20% |
| Cr | 10-40% | 0-40% | 0-40% | 0-40% |
| Cu | 0-20% | 0-20% | 0-20% | 0-20% |
| Co | 10-60% | 0-60% | 0-60% | 0-60% |
| Fe | 0-80% | 30-80% | 0-80% | 0-70% |
| Hf | 0-20% | 0-20% | 0-20% | 0-20% |
| Ir | 0-20% | 0-20% | 0-20% | 0-20% |
| Mg | 0-20% | 0-20% | 0-20% | 0-20% |
| Mn | 0-20% | 0-40% | 0-20% | 0-20% |
| Mo | 0-60% | 0-60% | 0-80% | 0-70% |
| Nb | 0-60% | 0-60% | 0-65% | 20-60% |
| Ni | 0-60% | 5-55% | 0-52% | 0-50% |
| Os | 0-20% | 0-20% | 0-20% | 0-20% |
| Pt | 0-20% | 0-20% | 0-20% | 0-20% |
| Re | 4.5-98% | 4.5-90% | 4.5-80% | 4.5-70% |
| Rh | 0-20% | 0-20% | 0-20% | 0-20% |
| Si | 0-20% | 0-20% | 0-20% | 0-20% |
| Sn | 0-20% | 0-20% | 0-20% | 0-20% |
| Ta | 0-60% | 0-60% | 5-65% | 0-60% |
| Tc | 0-20% | 0-20% | 0-20% | 0-20% |
| Ti | 0-60% | 0-55% | 0-53% | 0-50% |
| V | 0-20% | 0-20% | 2-20% | 0-20% |
| W | 0-60% | 0-60% | 0-80% | 0-70% |
| Y | 0-20% | 0-20% | 0-20% | 0-20% |
| Zr | 0-20% | 0-20% | 0-20% | 5-20% |

| | Component/Wt. % | | | |
|---|---|---|---|---|
| | Ex. 9 | Ex. 10 | Ex. 11 | Ex. 12 |
| Ag | 0-5% | 0-5% | 0-5% | 0-5% |
| Al | 0-5% | 0-5% | 1-15% | 0-20% |
| Bi | 0-5% | 0-5% | 0-5% | 0-5% |
| Cr | 1-28% | 1-30% | 0-5% | 0-30% |
| Cu | 0-20% | 0-5% | 0-5% | 0-25% |
| Co | 0-5% | 1-60% | 0-5% | 0-60% |
| Fe | 10-80% | 0-25% | 0-5% | 0-80% |
| Hf | 0-5% | 0-5% | 0-5% | 0-5% |
| Ir | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-8% | 0-25% | 0-5% | 0-98% |
| Nb | 0-5% | 0-5% | 0-5% | 0-95% |
| Ni | 1-20% | 1-45% | 0-5% | 0-50% |
| Os | 0-5% | 0-5% | 0-5% | 0-5% |
| Pt | 0-5% | 0-5% | 0-5% | 0-5% |
| Re | 5-20% | 4.8-20% | 4.5-20% | 4.5-20% |
| Rh | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 0-5% | 0-98% |
| Tc | 0-5% | 0-5% | 0-5% | 0-5% |
| Ti | 0-5% | 0-5% | 40-93% | 0-93% |
| V | 0-5% | 0-5% | 1-10% | 0-20% |
| W | 0-5% | 0-20% | 0-5% | 0-98% |
| Y | 0-5% | 0-5% | 0-5% | 0-5% |
| Zr | 0-5% | 0-5% | 0-5% | 0-5% |

| | Component/Wt. % | | | |
|---|---|---|---|---|
| | Ex. 13 | Ex. 14 | Ex. 15 | Ex. 16 |
| Mo | 30-80% | 35-80% | 30-70% | 35-65% |
| Hf | 0.8-1.4% | 0-2% | 0-2.5% | 0-2.5% |
| Re | 7-49% | 7-49% | 7-60% | 7.5-49% |
| Ta | 0-2% | 0-2% | 0-50% | 0-50% |
| W | 0-2% | 0-2% | 0-50% | 20-50% |

| | Component/Wt. % | | | |
|---|---|---|---|---|
| | Ex. 17 | Ex. 18 | Ex. 10 | Ex. 20 |
| W | 20-93% | 60-92% | 20-75% | 5-98% |
| Re | 6-60% | 8-40% | 7.5-47.5% | 0-80% |
| Mo | 0-47.5% | <0.5% | 1-47.5% | 0-80% |

| | Component/Wt. % | | | |
|---|---|---|---|---|
| | Ex. 21 | Ex. 22 | Ex. 23 | Ex. 24 |
| Re | 5-60% | 5-60% | 5-60% | 5-60% |
| Mo | 0-55% | 10-55% | 10-55% | 10-55% |
| Bi | 1-42 | 0-32 | 0-32 | 0-32 |
| Cr | 0-32 | 1-42 | 0-32 | 0-32 |
| Ir | 0-32 | 0-32 | 1-42 | 0-32 |
| Nb | 0-32 | 0-32 | 0-32 | 1-42 |
| Ta | 0-32 | 0-32 | 0-32 | 0-32 |
| Ti | 0-32 | 0-32 | 0-32 | 0-32 |
| Y | 0-32 | 0-32 | 0-32 | 0-32 |
| Zr | 0-32 | 0-32 | 0-32 | 0-32 |

| | Component/Wt. % | | | |
|---|---|---|---|---|
| | Ex. 25 | Ex. 26 | Ex. 27 | Ex. 28 |
| Re | 5-60% | 5-60% | 5-60% | 5-60% |
| Mo | 15-55% | 15-55% | 15-55% | 15-55% |
| Bi | 0-32 | 0-32 | 0-32 | 0-32 |
| Cr | 0-32 | 0-32 | 0-32 | 0-32 |
| Ir | 0-32 | 0-32 | 0-32 | 0-32 |
| Nb | 0-32 | 0-32 | 0-32 | 0-32 |
| Ta | 1-42 | 0-32 | 0-32 | 0-32 |
| Ti | 0-32 | 1-42 | 0-32 | 0-32 |
| Y | 0-32 | 0-32 | 1-42 | 0-32 |
| Zr | 0-32 | 0-32 | 0-32 | 1-42 |

| | Component/Wt. % | | | |
|---|---|---|---|---|
| | Ex. 29 | Ex. 30 | Ex. 31 | Ex. 32 |
| Re | 50-75% | 55-75% | 60-75% | 65-75% |
| Cr | 25-50% | 25-45% | 25-40% | 25-35% |
| Mo | 0-25% | 0-25% | 0-25% | 0-25% |
| Bi | 0-25% | 0-25% | 0-25% | 0-25% |
| Ir | 0-25% | 0-25% | 0-25% | 0-25% |
| Nb | 0-25% | 0-25% | 0-25% | 0-25% |
| Ta | 0-25% | 0-25% | 0-25% | 0-25% |
| V | 0-25% | 0-25% | 0-25% | 0-25% |
| W | 0-25% | 0-25% | 0-25% | 0-25% |
| Mn | 0-25% | 0-25% | 0-25% | 0-25% |
| Tc | 0-25% | 0-25% | 0-25% | 0-25% |
| Ru | 0-25% | 0-25% | 0-25% | 0-25% |
| Rh | 0-25% | 0-25% | 0-25% | 0-25% |
| Hf | 0-25% | 0-25% | 0-25% | 0-25% |
| Os | 0-25% | 0-25% | 0-25% | 0-25% |
| Cu | 0-25% | 0-25% | 0-25% | 0-25% |
| Ir | 0-25% | 0-25% | 0-25% | 0-25% |
| Ti | 0-25% | 0-25% | 0-25% | 0-25% |
| Y | 0-25% | 0-25% | 0-25% | 0-25% |
| Zr | 0-25% | 0-25% | 0-25% | 0-25% |
| Ag | 0-25% | 0-25% | 0-25% | 0-25% |
| Al | 0-25% | 0-25% | 0-25% | 0-22% |
| Co | 0-25% | 0-25% | 0-25% | 0-25% |
| Fe | 0-25% | 0-25% | 0-25% | 0-25% |
| Mg | 0-25% | 0-25% | 0-25% | 0-25% |
| Ni | 0-25% | 0-25% | 0-25% | 0-25% |
| Pt | 0-25% | 0-25% | 0-25% | 0-25% |
| Si | 0-25% | 0-25% | 0-25% | 0-25% |
| Sn | 0-25% | 0-25% | 0-25% | 0-25% |

| | Component/Wt. % | | | |
|---|---|---|---|---|
| | Ex. 33 | Ex. 34 | Ex. 35 | Ex. 36 |
| Re | 50-75% | 55-72% | 60-70% | 62-70% |
| Cr | 24-49% | 27-44% | 29-39% | 29-37% |
| Mo | 1-15% | 1-10% | 1-8% | 1-5% |
| Bi | 0-15% | 0-10% | 0-8% | 0-5% |
| Ir | 0-15% | 0-10% | 0-8% | 0-5% |
| Nb | 0-15% | 0-10% | 0-8% | 0-5% |
| Ta | 0-15% | 0-10% | 0-8% | 0-5% |
| V | 0-15% | 0-10% | 0-8% | 0-5% |
| W | 0-15% | 0-10% | 0-8% | 0-5% |
| Mn | 0-15% | 0-10% | 0-8% | 0-5% |
| Tc | 0-15% | 0-10% | 0-8% | 0-5% |
| Ru | 0-15% | 0-10% | 0-8% | 0-5% |
| Rh | 0-15% | 0-10% | 0-8% | 0-5% |
| Hf | 0-15% | 0-10% | 0-8% | 0-5% |
| Os | 0-15% | 0-10% | 0-8% | 0-5% |
| Cu | 0-15% | 0-10% | 0-8% | 0-5% |
| Ir | 0-15% | 0-10% | 0-8% | 0-5% |
| Ti | 0-15% | 0-10% | 0-8% | 0-5% |
| Y | 0-15% | 0-10% | 0-8% | 0-5% |
| Zr | 0-15% | 0-10% | 0-8% | 0-5% |
| Ag | 0-15% | 0-10% | 0-8% | 0-5% |
| Al | 0-15% | 0-10% | 0-8% | 0-5% |
| Co | 0-15% | 0-10% | 0-8% | 0-5% |
| Fe | 0-15% | 0-10% | 0-8% | 0-5% |
| Mg | 0-15% | 0-10% | 0-8% | 0-5% |
| Ni | 0-15% | 0-10% | 0-8% | 0-5% |
| Pt | 0-15% | 0-10% | 0-8% | 0-5% |
| Si | 0-15% | 0-10% | 0-8% | 0-5% |
| Sn | 0-15% | 0-10% | 0-8% | 0-5% |

| | Component/Wt. % | | | |
|---|---|---|---|---|
| | Ex. 37 | Ex. 38 | Ex. 39 | Ex. 40 |
| Mo | 40-95% | 40-95% | 40-95% | 40-95% |
| Co | ≤0.002% | ≤0.002% | ≤0.002% | ≤0.002% |
| Fe | ≤0.02% | ≤0.02% | ≤0.02% | ≤0.02% |
| Hf | 0.1-2.5% | 0-2.5% | 0-2.5% | 0-2.5% |
| Os | ≤1% | ≤1% | ≤1% | ≤1% |
| Nb | ≤0.01% | ≤0.01% | ≤0.01% | ≤0.01% |
| Pt | ≤1% | ≤1% | ≤1% | ≤1% |
| Re | 5-49% | 5-49% | 5-49% | 5-49% |
| Sn | ≤0.002% | ≤0.002% | ≤0.002% | ≤0.002% |
| Ta | 0-50% | 0-50% | 0-50% | 0-50% |
| Tc | ≤1% | ≤1% | ≤1% | ≤1% |
| Ti | ≤1% | ≤1% | ≤1% | ≤1% |
| V | ≤1% | ≤1% | ≤1% | ≤1% |
| W | 0-50% | 0-50% | 0-50% | 0.5-50% |
| Zr | ≤1% | ≤1% | ≤1% | ≤1% |
| Ag | 0-5% | 0-5% | 0-5% | 0-5% |
| Al | 0-5% | 0-5% | 0-5% | 0-5% |
| Co | 0-5% | 0-5% | 0-5% | 0-5% |
| Mg | 0-5% | 0-5% | 0-5% | 0-5% |
| Ni | 0-5% | 0-5% | 0-5% | 0-5% |
| Si | 0-5% | 0-5% | 0-5% | 0-5% |
| Sn | 0-5% | 0-5% | 0-5% | 0-5% |

| | Component/Wt. % | | |
|---|---|---|---|
| | Ex. 41 | Ex. 42 | Ex. 43 |
| W | 20-95% | 60-95% | 20-80% |
| Re | 5-47.5% | 5-40% | 5-47.5% |
| Mo | 0-47.5% | <0.5% | 1-47.5% |
| Cu | <0.5% | <0.5% | <0.5% |
| Co | ≤0.002% | ≤0.002% | ≤0.002% |
| Fe | ≤0.02% | ≤0.02% | ≤0.02% |
| Hf | <0.5% | <0.5% | <0.5% |
| Os | <0.5% | <0.5% | <0.5% |
| Nb | ≤0.01% | ≤0.01% | ≤0.01% |
| Pt | <0.5% | <0.5% | <0.5% |
| Sn | ≤0.002% | ≤0.002% | ≤0.002% |
| Ta | <0.5% | <0.5% | <0.5% |
| To | <0.5% | <0.5% | <0.5% |
| Ti | <0.5% | <0.5% | <0.5% |
| V | <0.5% | <0.5% | <0.5% |
| Zr | <0.5% | <0.5% | <0.5% |
| Ag | 0-5% | 0-5% | 0-5% |
| Al | 0-5% | 0-5% | 0-5% |
| Fe | 0-5% | 0-5% | 0-5% |
| Mg | 0-5% | 0-5% | 0-5% |
| Ni | 0-5% | 0-5% | 0-5% |
| Si | 0-5% | 0-5% | 0-5% |

| | Component/Wt. % | | | |
|---|---|---|---|---|
| | Ex. 44 | Ex. 45 | Ex. 46 | Ex. 47 |
| W | 1-94.9% | 1-94.9% | 1-94.9% | 10-95% |
| Cu | 0.1-94% | 0.1-94% | 0.1-94% | 1-84% |
| Co | ≤0.002% | ≤0.002% | ≤0.002% | ≤0.002% |
| Fe | ≤0.02% | ≤0.02% | ≤0.02% | ≤0.02% |
| Hf | 0.1-2.5% | 0-2.5% | 0-2.5% | 0-2.5% |
| Os | ≤1% | ≤1% | ≤1% | ≤1% |
| Mo | 0-5% | 0.1-3% | 0-2% | 0-3% |
| Nb | ≤0.01% | ≤0.01% | ≤0.01% | ≤0.01% |
| Pt | ≤1% | ≤1% | ≤1% | ≤1% |
| Re | 5-40% | 5-40% | 5-40% | 6-40% |
| Sn | ≤0.002% | ≤0.002% | ≤0.002% | ≤0.002% |
| Ta | 0-50% | 0-50% | 0-50% | 0-50% |
| Tc | ≤1% | ≤1% | ≤1% | ≤1% |
| Ti | ≤1% | ≤1% | ≤1% | ≤1% |
| V | ≤1% | ≤1% | ≤1% | ≤1% |
| Zr | ≤1% | ≤1% | ≤1% | ≤1% |
| Ag | 0-5% | 0-5% | 0-5% | 0-5% |
| Al | 0-5% | 0-5% | 0-5% | 0-5% |
| Fe | 0-5% | 0-5% | 0-5% | 0-5% |
| Mg | 0-5% | 0-5% | 0-5% | 0-5% |
| Ni | 0-5% | 0-5% | 0-5% | 0-5% |
| Si | 0-5% | 0-5% | 0-5% | 0-5% |

| | Component/Wt. % | | |
|---|---|---|---|
| | Ex. 48 | Ex. 49 | Ex. 50 |
| W | 20-96% | 25-92% | 30-88% |
| Cu | 2-74% | 2-68% | 5-62% |
| Co | ≤0.002% | ≤0.002% | ≤0.002% |
| Hf | 0-2.5% | 0-2.5% | 0-2.5% |
| Os | ≤1% | ≤1% | ≤1% |
| Mo | 0-3% | 0-2% | 0-1% |
| Nb | ≤0.01% | ≤0.01% | ≤0.01% |
| Pt | ≤1% | ≤1% | ≤1% |
| Re | 6-40% | 7-40% | 8-40% |
| Sn | ≤0.002% | ≤0.002% | ≤0.002% |
| Ta | 0-50% | 0.5-50% | 0-50% |
| Tc | ≤1% | ≤1% | ≤1% |
| Ti | ≤1% | ≤1% | ≤1% |
| V | ≤1% | ≤1% | ≤1% |
| Ag | 0-5% | 0-5% | 0-5% |
| Al | 0-5% | 0-5% | 0-5% |
| Fe | 0-5% | 0-5% | 0-5% |
| Mg | 0-5% | 0-5% | 0-5% |
| Ni | 0-5% | 0-5% | 0-5% |
| Si | 0-5% | 0-5% | 0-5% |

| Component/Wt. % | Ex. 51 | Ex. 52 | Ex. 53 | Ex. 54 |
|---|---|---|---|---|
| W | 25-88% | 35-87% | 40-86% | 50-85% |
| Cu | 5-68% | 5-57% | 5-51% | 5-40% |
| Hf | 0.8-1.4% | 0-2.5% | 0-2.5% | 0-2.5% |
| Re | 0-40% | 0-40% | 0-40% | 0-40% |
| Ta | 0-50% | 0-50% | 0-50% | 0-50% |

| Component/Wt. % | Ex. 55 | Ex. 56 | Ex. 57 |
|---|---|---|---|
| Ti | 55-66% | 65-76% | 70-76% |
| Mo | 20-41% | 20-31% | 20-26% |
| Re | 4-20% | 4-20% | 4-20% |
| Yt | <0.5% | <0.5% | <0.5% |
| Nb | <0.5% | <0.5% | <0.5% |
| Co | <0.5% | <0.5% | <0.5% |
| Cr | <0.5% | <0.5% | <0.5% |
| Zr | <0.5% | <0.5% | <0.5% |

| Component/Wt. % | Ex. 58 | Ex. 59 | Ex. 60 |
|---|---|---|---|
| W | 20-95% | 60-93% | 20-80% |
| Re | 5-47.5% | 7-40% | 5-47.5% |
| Mo | 0-47.5% | <0.5% | 1-47.5% |
| Cu | <0.5% | <0.5% | <0.5% |
| Co | ≤0.002% | ≤0.002% | ≤0.002% |
| Fe | ≤0.02% | ≤0.02% | ≤0.02% |
| Hf | <0.5% | <0.5% | <0.5% |
| Os | <0.5% | <0.5% | <0.5% |
| Nb | ≤0.01% | ≤0.01% | ≤0.01% |
| Pt | <0.5% | <0.5% | <0.5% |
| Sn | ≤0.002% | ≤0.002% | ≤0.002% |
| Ta | <0.5% | <0.5% | <0.5% |
| Tc | <0.5% | <0.5% | <0.5% |
| Ti | <0.5% | <0.5% | <0.5% |
| V | <0.5% | <0.5% | <0.5% |
| Zr | <0.5% | <0.5% | <0.5% |
| Ag | 0-5% | 0-5% | 0-5% |
| Al | 0-5% | 0-5% | 0-5% |
| Fe | 0-5% | 0-5% | 0-5% |
| Mg | 0-5% | 0-5% | 0-5% |
| Ni | 0-5% | 0-5% | 0-5% |
| Si | 0-5% | 0-5% | 0-5% |

| Component/Wt. % | Ex. 61 | Ex. 62 | Ex. 63 | Ex. 64 |
|---|---|---|---|---|
| Ag | 0-10% | 0-10% | 0-10% | 0-10% |
| Al | 0-10% | 0-10% | 0-10% | 2-10% |
| B | 0-10% | 0-10% | 0-10% | 0-10% |
| Bi | 0-10% | 0-10% | 0-10% | 0-10% |
| Cr | 2-30% | 10-30% | 0-20% | 0-20% |
| Cu | 0-10% | 0-10% | 0-10% | 0-10% |
| Co | 0-10% | 32-70% | 0-10% | 0-10% |
| Fe | 50-80% | 0-20% | 0-10% | 0-10% |
| Hf | 0-10% | 0-10% | 0-10% | 0-10% |
| Ir | 0-10% | 0-10% | 0-10% | 0-10% |
| La | 0-10% | 0-10% | 0-10% | 0-10% |
| Mg | 0-10% | 0-10% | 0-10% | 0-10% |
| Mn | 0-20% | 0-10% | 0-10% | 0-10% |
| Mo | 0-10% | 0-30% | 0-16% | 0-16% |
| Nb | 0-10% | 0-10% | 0-10% | 0-10% |
| Ni | 0.1-30% | 0.1-40% | 0-10% | 0-10% |
| Os | 0-10% | 0-10% | 0-10% | 0-10% |
| Pt | 0-10% | 0-10% | 0-10% | 0-10% |
| Re | 5-40% | 4.8-40% | 4.5-80% | 4.5-80% |
| Rh | 0-10% | 0-10% | 0-10% | 0-10% |
| Se | 0-10% | 0-10% | 0-10% | 0-10% |
| Si | 0-10% | 0-10% | 0-10% | 0-10% |
| Sn | 0-10% | 0-10% | 0-12% | 0-12% |
| Ta | 0-10% | 0-10% | 0-10% | 0-10% |
| Tc | 0-10% | 0-10% | 0-10% | 0-10% |
| Ti | 0-10% | 0-10% | 70-91.5% | 70-91.5% |
| V | 0-10% | 0-10% | 0-10% | 0.01-10% |
| W | 0-10% | 0-20% | 0-10% | 0-10% |
| Y | 0-10% | 0-10% | 0-10% | 0-10% |
| Zr | 0-10% | 0-10% | 0-10% | 0-10% |

| Component/Wt. % | Ex. 65 | Ex. 66 | Ex. 67 | Ex. 68 |
|---|---|---|---|---|
| Ag | 0-10% | 0-10% | 0-10% | 0-10% |
| Al | 0-10% | 0-10% | 0-10% | 0-10% |
| B | 0-10% | 0-10% | 0-10% | 0-10% |
| Bi | 0-10% | 0-10% | 0-10% | 0-10% |
| Cr | 0-10% | 0-20% | 0-20% | 0-10% |
| Cu | 0-10% | 0-10% | 0-50% | 0-10% |
| Co | 0-10% | 0-10% | 0-10% | 0-10% |
| Fe | 0-10% | 0-10% | 0-10% | 0-10% |
| Hf | 0-10% | 0-10% | 0-10% | 0-10% |
| Ir | 0-10% | 0-10% | 0-10% | 0-12% |
| La | 0-10% | 0-10% | 0-10% | 0-10% |
| Mg | 0-10% | 0-10% | 0-10% | 0-10% |
| Mn | 0-10% | 0-10% | 0-10% | 0-10% |
| Mo | 0-55% | 40-93% | 0-50% | 0-20% |
| Nb | 0-10% | 0-10% | 0-10% | 40-85% |
| Ni | 0-45% | 0-10% | 0-10% | 0-10% |
| Os | 0-10% | 0-10% | 0-10% | 0-10% |
| Pt | 0-10% | 0-10% | 0-10% | 0-10% |
| Re | 14-40% | 7-40% | 7-40% | 7-40% |
| Rh | 0-10% | 0-10% | 0-10% | 0-10% |
| Se | 0-10% | 0-10% | 0-10% | 0-10% |
| Si | 0-10% | 0-10% | 0-10% | 0-10% |
| Sn | 0-10% | 0-10% | 0-10% | 0-10% |
| Ta | 35-84% | 0-50% | 0-50% | 0-35% |
| Tc | 0-10% | 0-10% | 0-10% | 0-10% |
| Ti | 0-10% | 0-10% | 0-10% | 0-10% |
| V | 0-10% | 0-10% | 0-10% | 0-10% |
| W | 0.1-25% | 0-50% | 14-10% | 0-15% |
| Y | 0-10% | 0-10% | 0-10% | 0-10% |
| Zr | 0-10% | 0-10% | 0-50% | 0-10% |

| Component/Wt. % | Ex. 69 | Ex. 70 | Ex. 71 | Ex. 72 |
|---|---|---|---|---|
| Ag | 0-10% | 0-10% | 0-5% | 0-5% |
| Al | 0-10% | 0-10% | 0-5% | 5-7% |
| B | 0-10% | 0-10% | 0-5% | 0-5% |
| Bi | 0-10% | 0-10% | 0-5% | 0-5% |
| Cr | 0-10% | 1-95% | 12-28% | 0-5% |
| Cu | 0-10% | 0-10% | 0-5% | 0-5% |
| Co | 0-10% | 0-10% | 36-68% | 0-5% |
| Fe | 0-10% | 0-10% | 0-18% | 0-5% |
| Hf | 0-10% | 0-10% | 0-5% | 0-5% |
| Ir | 0-10% | 0-10% | 0-5% | 0-5% |
| La | 0-10% | 0-10% | 0-5% | 0-5% |
| Mg | 0-10% | 0-10% | 0-5% | 0-5% |
| Mn | 0-10% | 0-10% | 0-5% | 0-5% |
| Mo | 0-10% | 0-20% | 0-12% | 0-5% |
| Nb | 0-10% | 0-10% | 0-5% | 0-5% |
| Ni | 30-58% | 0-10% | 9-36% | 0-5% |
| Os | 0-10% | 0-10% | 0-5% | 0-5% |
| Pt | 0-10% | 0-10% | 0-5% | 0-5% |
| Re | 5-40% | 5-40% | 4.8-40% | 4.5-40% |
| Rh | 0-10% | 0-10% | 0-5% | 0-5% |
| Se | 0-10% | 0-10% | 0-5% | 0-5% |
| Si | 0-10% | 0-10% | 0-5% | 0-5% |
| Sn | 0-10% | 0-10% | 0-5% | 0-5% |
| Ta | 0-10% | 0-10% | 0-5% | 0-5% |
| Tc | 0-10% | 0-10% | 0-5% | 0-5% |
| Ti | 30-58% | 0-40% | 0-5% | 70-91.5% |
| V | 0-10% | 0-10% | 0-5% | 3-6% |
| W | 0-10% | 0-10% | 0-16% | 0-5% |
| Y | 0-10% | 0-10% | 0-5% | 0-5% |
| Zr | 0-10% | 0-20% | 0-5% | 0-5% |

| Component/Wt. % | Ex. 73 | Ex. 74 | Ex. 75 | Ex. 76 |
|---|---|---|---|---|
| Ag | 0-8% | 0-8% | 0-8% | 0-8% |
| Al | 0-8% | 0-8% | 0-8% | 2-10% |
| B | 0-8% | 0-8% | 0-8% | 0-8% |
| Bi | 0-8% | 0-8% | 0-8% | 0-8% |
| Cr | 2-30% | 10-30% | 0-20% | 0-20% |
| Cu | 0-8% | 0-8% | 0-8% | 0-8% |
| Co | 0-8% | 32-70% | 0-8% | 0-8% |
| Fe | 50-80% | 0-20% | 0-8% | 0-8% |
| Hf | 0-8% | 0-8% | 0-8% | 0-8% |
| Ir | 0-8% | 0-8% | 0-8% | 0-8% |
| La | 0-8% | 0-8% | 0-8% | 0-8% |
| Mg | 0-8% | 0-8% | 0-8% | 0-8% |
| Mn | 0-20% | 0-8% | 0-8% | 0-8% |
| Mo | 0-8% | 0-30% | 0-16% | 0-16% |
| Nb | 0-8% | 0-8% | 0-8% | 0-8% |
| Ni | 0.1-30% | 0.1-40% | 0-8% | 0-8% |
| Os | 0-8% | 0-8% | 0-8% | 0-8% |
| Pt | 0-8% | 0-8% | 0-8% | 0-8% |
| Re | 5-40% | 4.8-40% | 4.5-80% | 4.5-80% |
| Rh | 0-8% | 0-8% | 0-8% | 0-8% |
| Se | 0-8% | 0-8% | 0-8% | 0-8% |
| Si | 0-8% | 0-8% | 0-8% | 0-8% |
| Sn | 0-8% | 0-8% | 0-12% | 0-12% |
| Ta | 0-8% | 0-8% | 0-8% | 0-8% |
| To | 0-8% | 0-8% | 0-8% | 0-8% |
| Ti | 0-8% | 0-8% | 70-91.5% | 70-91.5% |
| V | 0-8% | 0-8% | 0-8% | 0.01-10% |
| W | 0-8% | 0-20% | 0-8% | 0-8% |
| Y | 0-8% | 0-8% | 0-8% | 0-8% |
| Zr | 0-8% | 0-8% | 0-8% | 0-8% |

| Component/Wt. % | Ex. 77 | Ex. 78 | Ex. 79 | Ex. 80 |
|---|---|---|---|---|
| Ag | 0-8% | 0-8% | 0-8% | 0-8% |
| Al | 0-8% | 0-8% | 0-8% | 0-8% |
| B | 0-8% | 0-8% | 0-8% | 0-8% |
| Bi | 0-8% | 0-8% | 0-8% | 0-8% |
| Cr | 0-8% | 0-20% | 0-20% | 0-8% |
| Cu | 0-8% | 0-8% | 0-50% | 0-8% |
| Co | 0-8% | 0-8% | 0-8% | 0-8% |
| Fe | 0-8% | 0-8% | 0-8% | 0-8% |
| Hf | 0-8% | 0-8% | 0-8% | 0-8% |
| Ir | 0-8% | 0-8% | 0-8% | 0-12% |
| La | 0-8% | 0-8% | 0-8% | 0-8% |
| Mg | 0-8% | 0-8% | 0-8% | 0-8% |
| Mn | 0-8% | 0-8% | 0-8% | 0-8% |
| Mo | 0-55% | 40-93% | 0-50% | 0-20% |
| Nb | 0-8% | 0-8% | 0-8% | 40-85% |
| Ni | 0-45% | 0-8% | 0-8% | 0-8% |
| Os | 0-8% | 0-8% | 0-8% | 0-8% |
| Pt | 0-8% | 0-8% | 0-8% | 0-8% |
| Re | 14-40% | 7-40% | 7-40% | 7-40% |
| Rh | 0-8% | 0-8% | 0-8% | 0-8% |
| Se | 0-8% | 0-8% | 0-8% | 0-8% |
| Si | 0-8% | 0-8% | 0-8% | 0-8% |
| Sn | 0-8% | 0-8% | 0-8% | 0-8% |
| Ta | 35-84% | 0-50% | 0-50% | 0-35% |
| Tc | 0-8% | 0-8% | 0-8% | 0-8% |
| Ti | 0-8% | 0-8% | 0-8% | 0-8% |
| V | 0-8% | 0-8% | 0-8% | 0-8% |
| W | 0.1-25% | 0-50% | 14-10% | 0-15% |
| Y | 0-8% | 0-8% | 0-8% | 0-8% |
| Zr | 0-8% | 0-8% | 0-50% | 0-8% |

| Component/Wt. % | Ex. 81 | Ex. 82 | Ex. 83 | Ex. 84 |
|---|---|---|---|---|
| Ag | 0-5% | 0-5% | 0-5% | 0-5% |
| Al | 0-5% | 0-5% | 0-5% | 5-7% |
| B | 0-5% | 0-5% | 0-5% | 0-5% |
| Bi | 0-5% | 0-5% | 0-5% | 0-5% |
| Cr | 0-5% | 1-95% | 12-28% | 0-5% |
| Cu | 0-5% | 0-5% | 0-5% | 0-5% |
| Co | 0-5% | 0-5% | 36-68% | 0-5% |
| Fe | 0-5% | 0-5% | 0-18% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-5% | 0-20% | 0-12% | 0-5% |
| Nb | 0-5% | 0-5% | 0-5% | 0-5% |
| Ni | 30-58% | 0-5% | 9-36% | 0-5% |
| Os | 0-5% | 0-5% | 0-5% | 0-5% |
| Pt | 0-5% | 0-5% | 0-5% | 0-5% |
| Re | 5-40% | 5-40% | 4.8-40% | 4.5-40% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 0-5% | 0-5% |
| To | 0-5% | 0-5% | 0-5% | 0-5% |
| Ti | 30-58% | 0-40% | 0-5% | 70-91.5% |
| V | 0-5% | 0-5% | 0-5% | 3-6% |
| W | 0-5% | 0-5% | 0-16% | 0-5% |
| Y | 0-5% | 0-5% | 0-5% | 0-5% |
| Zr | 0-5% | 0-20% | 0-5% | 0-5% |

| Component/Wt. % | Ex. 85 | Ex. 86 | Ex. 87 | Ex. 88 |
|---|---|---|---|---|
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 0-5% | 0-5% | 0-5% |
| Cu | 0-5% | 0-5% | 0-5% | 0-5% |
| Co | 0-5% | 0-5% | 0-5% | 0-5% |
| Fe | 0-5% | 0-5% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 1-15% | 2-10% | 3-8% | 0-5% |
| Nb | 0-5% | 0-5% | 0-5% | 20-45% |
| Ni | 0-5% | 0-5% | 0-5% | 0-5% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 0-5% | 1-15% |
| Tc | 0-5% | 0-5% | 0-5% | 0-5% |
| Ti | 51-70% | 51-70% | 55-70% | 51-70% |
| V | 0-5% | 0-5% | 0-5% | 0-5% |
| W | 0-5% | 0-5% | 0-5% | 0-5% |
| Y | 0-5% | 0-5% | 0-5% | 0-5% |
| Zr | 20-40% | 22-38% | 27-33% | 1-15% |

| Component/Wt. % | Ex. 89 | Ex. 90 | Ex. 91 | Ex. 92 |
|---|---|---|---|---|
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 0-5% | 0-5% | 0-5% |
| Cu | 0-5% | 0-5% | 0-5% | 0-5% |
| Co | 0-5% | 0-5% | 0-5% | 0-5% |
| Fe | 0-5% | 0-5% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-5% | 0-5% | 0-5% | 0-5% |
| Nb | 25-40% | 30-40% | 25-40% | 26-32% |
| Ni | 0-5% | 0-5% | 0-5% | 0-5% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 2-8% | 3-6% | 5-15% | 10-14% |
| Tc | 0-5% | 0-5% | 0-5% | 0-5% |
| Ti | 51-70% | 52-63% | 51-68% | 51-62% |
| V | 0-5% | 0-5% | 0-5% | 0-5% |
| W | 0-5% | 0-5% | 0-5% | 0-5% |
| Y | 0-5% | 0-5% | 0-5% | 0-5% |
| Zr | 2-12% | 4-8% | 2-8% | 2-6% |

| Component/Wt. % | Ex. 93 | Ex. 94 | Ex. 95 | Ex. 96 |
|---|---|---|---|---|
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 5-35% | 10-30% | 15-25% |
| Cu | 0-5% | 0-5% | 0-5% | 0-5% |
| Co | 0-5% | 20-55% | 25-50% | 35-45% |
| Fe | 0-5% | 3-25% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-5% | 2-15% | 3-12% | 4-9% |
| Nb | 30-40% | 0-5% | 0-5% | 0-5% |
| Ni | 0-5% | 4-23% | 5-20% | 10-18% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 1-3% | 0-5% | 0-5% | 0-5% |
| Tc | 0-5% | 0-5% | 0-5% | 0-5% |
| Ti | 51-67% | 0-5% | 0-5% | 0-5% |
| V | 0-5% | 0-5% | 0-5% | 0-5% |
| W | 0-5% | 0-5% | 0-5% | 0-5% |
| Y | 0-5% | 0-5% | 0-5% | 0-5% |
| Zr | 2-5% | 0-5% | 0-5% | 0-5% |

| Component/Wt. % | Ex. 97 | Ex. 98 | Ex. 99 | Ex. 100 |
|---|---|---|---|---|
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 0-5% | 0-5% | 0-5% |
| Cu | 0-5% | 0-5% | 0-5% | 0-5% |
| Co | 0-5% | 0-5% | 0-5% | 0-5% |
| Fe | 0-5% | 0-5% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 30-65% | 40-60% | 45-55% | 0-5% |
| Nb | 0-5% | 0-5% | 0-5% | 55-99.75% |
| Ni | 0-5% | 0-5% | 0-5% | 0-5% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 0-5% | 0-5% |
| Tc | 0-5% | 0-5% | 0-5% | 0-5% |
| Ti | 0-5% | 0-5% | 0-5% | 0-5% |
| V | 0-5% | 0-5% | 0-5% | 0-5% |
| W | 0-5% | 0-5% | 0-5% | 0-5% |
| Y | 0-5% | 0-5% | 0-5% | 0-5% |
| Zr | 30-56% | 40-60% | 45-55% | 0.25-45% |

| Component/ | | | | |
|---|---|---|---|---|
| Wt. % | Ex. 101 | Ex. 102 | Ex. 103 | Ex. 104 |
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 0-5% | 0-5% | 0-5% |
| Cu | 0-5% | 0-5% | 0-5% | 0-5% |
| Co | 0-5% | 0-5% | 0-5% | 0-5% |
| Fe | 0-5% | 0-5% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-5% | 0-5% | 0-5% | 0-5% |
| Nb | 75-99.5% | 95-99.25% | 55-78.5% | 68-74.25% |
| Ni | 0-5% | 0-5% | 0-5% | 0-5% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 20-35% | 25-30% |
| Tc | 0-5% | 0-5% | 0-5% | 0-5% |
| Ti | 0-5% | 0-5% | 0-5% | 0-5% |
| V | 0-5% | 0-5% | 0-5% | 0-5% |
| W | 0-5% | 0-5% | 1-8% | 0-5% |
| Y | 0-5% | 0-5% | 0-5% | 0-5% |
| Zr | 0.5-25% | 0.75-5% | 0.5-5% | 0.75-3% |

| Element/Wt. % | Ex. 105 | Ex. 106 | Ex. 107 | Ex. 108 |
|---|---|---|---|---|
| Re | 30-75% | 40-75% | 45-75% | 45-70% |
| Cr | 25-70% | 25-65% | 25-55% | 30-55% |
| Mo | 0-25% | 0-25% | 1-25% | 2-25% |
| Bi | 0-25% | 0-25% | 0-25% | 0-25% |
| Cr | 0-25% | 0-25% | 0-25% | 0-25% |
| Ir | 0-25% | 0-25% | 0-25% | 0-25% |
| Nb | 0-25% | 0-25% | 0-25% | 0-25% |
| Ta | 0-25% | 0-25% | 0-25% | 0-25% |
| V | 0-25% | 0-25% | 0-25% | 0-25% |
| W | 0-25% | 0-25% | 0-25% | 0-25% |
| Mn | 0-25% | 0-25% | 0-25% | 0-25% |
| Tc | 0-25% | 0-25% | 0-25% | 0-25% |
| Ru | 0-25% | 0-25% | 0-25% | 0-25% |
| Rh | 0-25% | 0-25% | 0-25% | 0-25% |
| Hf | 0-25% | 0-25% | 0-25% | 0-25% |
| Os | 0-25% | 0-25% | 0-25% | 0-25% |
| Cu | 0-25% | 0-25% | 0-25% | 0-25% |
| Ir | 0-25% | 0-25% | 0-25% | 0-25% |
| Ti | 0-25% | 0-25% | 0-25% | 0-25% |
| Y | 0-25% | 0-25% | 0-25% | 0-25% |
| Zr | 0-25% | 0-25% | 0-25% | 0-25% |

In Examples 1-108, it will be appreciated that all of the above ranges include any value between the range and any other range that is between the ranges set forth above. Any of the above values that include the ≤ symbol includes the range from 0 to the stated value and all values and ranges therebetween.

In another and/or alternative non-limiting aspect of the present disclosure, the metal alloy that is used to partially or fully formed the frame of the prosthetic heart valve includes less than about 5 wt. % (e.g., 0-4.999999 wt. % and all values and ranges therebetween) other metals and/or impurities, typically 0-1 wt. %, more typically 0-0.1 wt. %, even more typically 0-0.01 wt. %, and still even more typically 0-0.001 wt. %. A high purity level of the metal alloy can result in the formation of a more homogeneous alloy, which in turn can result in a more uniform density throughout the metal alloy, and also can result in the desired yield and ultimate tensile strengths of the metal alloy.

In accordance with another and/or alternative aspect of the present disclosure, the frame for a prosthetic heart valve is optionally subjected to one or more manufacturing processes. These manufacturing processes can include, but are not limited to, expansion, laser cutting, etching, crimping, annealing, drawing, pilgering, electroplating, electro-polishing, machining, plasma coating, 3D printed coatings, chemical vapor deposition, chemical polishing, cleaning, pickling, ion beam deposition or implantation, sputter coating, vacuum deposition, etc.

In accordance with another and/or alternative aspect of the present disclosure, the metal alloy optionally includes a certain amount of carbon and oxygen; however, this is not required. These two elements have been found to affect the forming properties and brittleness of the metal alloy. The controlled atomic ratio of carbon and oxygen of the metal alloy can also minimize the tendency of the metal alloy to form micro-cracks during the forming of the metal alloy into a frame for a prosthetic heart valve, and/or during the use and/or expansion of the frame for a prosthetic heart valve in a body. The carbon to oxygen atomic ratio can be as low as about 0.2:1 (e.g., 0.2:1 to 50:1 and all values and ranges therebetween). In one non-limiting formulation, the carbon to oxygen atomic ratio in the metal alloy is generally at least about 0.3:1. Typically the carbon content of the metal alloy is less than about 0.1 wt. % (e.g., 0-0.0999999 wt. % and all values and ranges therebetween), and more typically 0-0.01 wt. %. Carbon contents that are too large can adversely affect the physical properties of the metal alloy. Generally, the oxygen content is to be maintained at very low level. In one non-limiting formulation, the oxygen content is less than about 0.1 wt. % of the metal alloy (e.g., 0-0.0999999 wt. % and all values and ranges therebetween), and typically 0-0.01 wt. %.

In accordance with another and/or alternative aspect of the present disclosure, the metal alloy optionally includes a controlled amount of nitrogen; however, this is not required. Large amounts of nitrogen in the metal alloy can adversely affect the ductility of the metal alloy. This can in turn adversely affect the elongation properties of the metal alloy. In one non-limiting formulation, the metal alloy includes less than about 0.001 wt. % nitrogen (e.g., 0 wt. % to 0.0009999 wt. % and all values and ranges therebetween). It is believed that the nitrogen content should be less than the content of carbon or oxygen in the metal alloy. In one non-limiting formulation, the atomic ratio of carbon to nitrogen is at least about 1.5:1 (e.g., 1.5:1 to 400:1 and all values and ranges therebetween). In another non-limiting formulation, the atomic ratio of oxygen to nitrogen is at least about 1.2:1 (e.g., 1.2:1 to 150:1 and all value and ranges therebetween).

In accordance with another and/or alternative aspect of the present disclosure, the metal alloy that is used to form all or part of the frame for a prosthetic heart valve 1) is not clad, metal coated, metal sprayed, plated and/or formed (e.g., cold worked, hot worked, etc.) onto another metal, or 2) does not have another metal or metal alloy metal sprayed, coated, plated, clad and/or formed onto the metal alloy. It will be appreciated that in some applications, the metal alloy of the present disclosure may be clad, metal sprayed, coated, plated and/or formed onto another metal, or another metal or metal alloy may be plated, metal sprayed, coated, clad and/or formed onto the metal alloy when forming all or a portion of a frame for a prosthetic heart valve.

In accordance with another and/or alternative aspect of the present disclosure, the metal alloy can be used to form a) a coating (e.g., cladding, dip coating, spray coating, plated coating, welded coating, plasma coating, etc.) on a portion of all of a frame for a prosthetic heart valve, or b) a core of a portion or all of a frame for a prosthetic heart valve. The composition of the coating is different from the composition of the material surface to which the metal alloy is coated. The coating thickness of the metal alloy is non-limiting (e.g., 1 μm to 1 inch and all values and ranges therebetween). In one non-limiting example, there is provided a frame for a prosthetic heart valve wherein a core or base layer of the frame for a prosthetic heart valve is formed of a metal or metal alloy (e.g., chromium alloy, titanium, titanium alloy, stainless steel, iron alloy, CoCr alloy, rhenium alloy, molybdenum alloy, tungsten alloy, Ta—W alloy, refractory metal alloy, MoTa alloy, MoRe alloy, etc.) or polymer or ceramic or composite material, and the other layer of the coated frame for a prosthetic heart valve is formed of a different metal or metal alloy. The core or base layer and the other layer of the frame for a prosthetic heart valve can each form 10-99% (and all values and ranges therebetween) of the overall cross section of the frame for a prosthetic heart valve. When the outer metal coating is a rhenium containing alloy, such rhenium alloy can be used to create a hard surface on the frame for a prosthetic heart valve at specific locations as well as all over the surface. In another non-limiting embodiment, the core or base layer of the frame for a prosthetic heart valve can be formed of a rhenium containing alloy and the coating layer includes one or more other materials (e.g., another type of metal or metal alloy [e.g., chromium alloy, titanium, titanium alloy, stainless steel, iron alloy, CoCr alloy, rhenium alloy, molybdenum alloy, tungsten alloy, Ta—W alloy, refractory metal alloy, MoTa alloy, MoRe alloy, etc.), polymer coating, ceramic coating, composite material coating, etc.). Non-limiting benefits of using the rhenium containing alloy in the core or interior layer of the frame for a prosthetic heart valve can include reducing the size of the frame for a prosthetic heart valve, increasing the strength of the frame for a prosthetic heart valve, and/or maintaining or reducing the cost of the frame for a prosthetic heart valve. As can be appreciated, the use of the rhenium containing alloy can result in other or additional advantages. The core or base layer size and/or thickness of the metal alloy are non-limiting. In one non-limiting example, there is provided a frame for a prosthetic heart valve that is at least partially formed from layered materials wherein a top layer is formed of material that is different form one or more other layers and the rhenium containing alloy forms one of the layers below the top layer, and the top layer is formed of a metal that is different from the rhenium containing alloy (e.g., chromium alloy, titanium, titanium alloy, stainless steel, iron alloy, CoCr alloy, rhenium alloy, molybdenum alloy, tungsten alloy, Ta—W alloy, refractory metal alloy, MoTa alloy, MoRe alloy, etc.). The core or lower layer or base layer and the outer layer of the layered material can each form 10-99% (and all values and ranges therebetween) of the overall cross section of the layered material.

In another and/or alternative non-limiting embodiment of the disclosure, the average tensile elongation of the metal alloy used to at least partially form the frame for a prosthetic heart valve is optionally at least about 20% (e.g., 20-50% average tensile elongation and all values and ranges therebetween). An average tensile elongation of at least 20% for the metal alloy is useful to facilitate in the frame for a prosthetic heart valve being properly expanded when positioned in the treatment area of a body. The desired tensile elongation can be obtained from a unique combination of the metals in the metal alloy in combination with achieving the desired purity and composition of the alloy and the desired grain size of the metal alloy.

In accordance with another and/or alternative aspect of the present disclosure, the metal alloy is optionally at least partially formed by a swaging process; however, this is not required. In one non-limiting embodiment, swaging is performed on the metal alloy to at least partially or fully achieve final dimensions of one or more portions of the frame for a prosthetic heart valve. The swaging dies can be shaped to fit the final dimension of the frame for a prosthetic heart valve; however, this is not required.

In accordance with another and/or alternative aspect of the present disclosure, the metal alloy can optionally be nitrided; however, this is not required. The nitrided layer on the metal alloy can function as a lubricating surface during the optional drawing of the metal alloy when partially or fully forming the frame for a prosthetic heart valve.

In accordance with another and/or alternative non-limiting aspect of the present disclosure, the frame of the prosthetic heart valve can be at least partially (e.g., 1-99.999 wt. % and all values and ranges therebetween) or fully formed from by 3D printing.

In accordance with another and/or alternative non-limiting aspect of the present disclosure, the average grain size of the metal alloy that is used to at least partially or fully form the frame of the prosthetic heart valve is optionally no greater than about 4 ASTM (e.g., 4 ASTM to 20 ASTM using ASTM E112 and all values and ranges therebetween, e.g., 0.35 micron to 90 micron, and all values and ranges therebetween). In another non-limiting embodiment of the disclosure, the average tensile elongation of the metal alloy used to partially or fully form the prosthetic heart valve is optionally at least about 25% (e.g., 25%-50% average tensile elongation and all values and ranges therebetween).

In accordance with another and/or alternative non-limiting aspect of the present disclosure, one or more components of the prosthetic heart valve (e.g., frame, inner skirt, outer skirt, leaflets, material used to secure leaflets to frame, etc.) can be partially (e.g., 1% to 99.99% and all values and ranges therebetween) or fully be coated with an enhancement coating to improve one or more properties of the prosthetic heat valve (e.g., change exterior color of material having coated surface, increase surface hardness by use of the coated surface, increase surface toughness material having coated surface, reduced friction via use of the coated surface, improve scratch resistance of material that has the coated surface, improve impact wear of coated surface, improve resistance to corrosion and oxidation of coated material, form a non-stick coated surface, improve biocompatibility of material having the coated surface, reduce toxicity of material having the coated surface, reduce ion release from material having the coated surface, the enhancement coating forms a surface that is less of an irritant to cell about the coated surface after the prosthetic heart valve is implanted, reduces the rate to which cells grown on coated surface after prosthetic heart valve is implanted, reduce rate to which leaflets fail to properly operate after prosthetic heart valve is implanted, etc.). In one non-limiting embodiment, only the frame of the prosthetic heart valve includes the enhancement coating, and wherein the frame is partially (e.g., 1-99.99% and all values and ranges therebetween) or fully coated with the enhancement coating. In another non-limiting embodiment, only one or more of all of the leaflets of the prosthetic heart valve include the enhancement coating, and wherein one or more or all of the leaflets are partially (e.g., 1-99.99% and all values and ranges therebetween) or fully coated with the enhancement coating. In another non-limiting embodiment, only the inner skirt of the prosthetic heart valve includes the enhancement coating, and wherein the inner skirt is partially (e.g., 1-99.99% and all values and ranges therebetween) or fully coated with the enhancement coating. In another non-limiting embodiment, only the outer skirt of the prosthetic heart valve includes the enhancement coating, and wherein the outer skirt is partially (e.g., 1-99.99% and all values and ranges therebetween) or fully coated with the enhancement coating. In another non-limiting embodiment, two or more or all of a) the frame, b) one or more or all of the leaflets, c) the inner skirt and d) the outer skirt of the prosthetic heart valve are partially (e.g., 1-99.99% and all values and ranges therebetween) or are fully coated with the enhancement coating. Non-limiting enhancement coatings that can be applied to a portion or all of the outer surface of one or more components of the prosthetic heart valve includes chromium nitride (CrN), diamond-like carbon (DLC), titanium nitride (TiN), titanium oxynitride or titanium nitride oxide (TiNOx), zirconium nitride (ZrN), zirconium oxide (ZrO₂), zirconium oxynitride (ZnNxOy) [e.g., cubic ZrN:O, cubic ZrO₂:N, tetragonal ZrO₂:N, and monoclinic ZrO₂:N phase coatings], oxyzirconium-nitrogen-carbon (ZrNC), zirconium OxyCarbide (ZrOC), and combinations of such coatings. In one non-limiting embodiment, the one or more enhancement coatings are optionally applied to a portion or all of the outer surface of one or more components of the prosthetic heart valve by a vacuum process using an energy source to vaporize material and deposit a thin layer of enhancement coating material. Such vacuum coating process, when used, can include a physical vapor deposition (PVD) process (e.g., sputter deposition, cathodic arc deposition or electron beam heating, etc.), chemical vapor deposition (CVD) process, atomic layer deposition (ALD) process, or a plasma-enhanced chemical vapor deposition (PE-CVD) process. In one non-limiting embodiment, the coating process is one or more of a PVD, CVD, ALD and PE-CVD, and wherein the coating process occurs at a temperature of 200-400° C. (and all values and ranges therebetween) for at least 10 minutes (e.g., 10-400 minutes and all values and ranges therebetween). In another non-limiting embodiment, the coating process is one or more of a PVD, CVD, ALD and PE-CVD, and wherein the coating process occurs at a temperature of 220-300° C. for 60-120 minutes. In another non-limiting embodiment, when the materials of the one or more enhancement coatings are to be applied to the outer surface of a frame of the prosthetic heart valve that is partially or fully formed of a metal alloy, the materials of the one or more enhancement coatings can optionally be combine with one or more metals in the metal alloy, and/or combined with nitrogen, oxygen, carbon, or other elements that are in the metal alloy and/or present in the atmosphere about the metal alloy to a form an enhancement coating on the outer surface of the metal alloy. In another non-limiting embodiment, when the materials of the one or more enhancement coatings are to be applied to the outer surface of a frame of the prosthetic heart valve that is partially or fully formed of a metal alloy, the materials of the one or more enhancement coatings can optionally be used to form various coating colors on the outer surface of the metal alloy (e.g., gold, copper, brass, black, rose gold, chrome, blue, silver, yellow, green, etc.). In another non-limiting embodiment, the thickness of the enhancement coating is greater than 1 nanometer (e.g., 2 nanometers to 100 microns and all values and ranges therebetween), and typically 0.1-25 microns, and more typically 0.2-10 microns. In another non-limiting embodiment, the hardness of the enhancement coating can be at least 5 GPa (ASTM C1327-15 or ASTM C1624-05), typically 5-50 GPa (and all values and ranges therebetween), more typically 10-25 GPa, and still more typically 14-24 GPa. In another non-limiting embodiment, the coefficient of friction (COF) of the enhancement coating can be 0.04-0.2 (and all values and ranges therebetween), and typically 0.6-0.15. In another non-limiting embodiment, the wear rate of the enhancement coating can be 0.5×10⁻⁷ mm³/N-m to 3×10⁻⁷ mm³/N-m (an all values and ranges therebetween), and typically 1.2×10⁻⁷ mm³/N-m to 2×10⁻⁷ mm³/N-m. In another non-limiting embodiment, silicon-based precursors (e.g., trimethysilane, tetramethylsilane, hexachlorodisilane, silane, dichlorosilane, trichlorosilane, silicon tetrachloride, tris(dimethylamino)silane, bis(tert-butylamino)silane, trisilylamine, allyltrimethoxysilane, (3-aminopropyl)triethoxysilane, butyltrichlorosilane, n-sec-butyl(trimethylsilyl)amine, chloropentamethyldisilane, 1,2-dichlorotetramethyldisilane, [3-(diethylamino)propyl]trimethoxysilane, 1,3-diethyl-1,1,3,3-tetramethyldisilazane, dimethoxydimethylsilane, dodecamethylcyclohexasilane, hexamethyldisilane, isobutyl(trimethoxy)silane, methyltrichlorosilane, 2,4,6,8,10-pentamethylcyclopentasiloxane, pentamethyldisilane, n-propyltriethoxysilane, silicon tetrabromide, silicon tetrabromide, etc.) can optionally be used to facilitate in the application of the enhancement coating to one or more portions or all of the outer surface of one or more components of the prosthetic heart valve.

In accordance with another and/or alternative non-limiting aspect of the present disclosure, one or more components of the prosthetic heart valve can be partially or fully coated with an enhancement coating composition that includes a chromium nitride (CrN) coating. A portion or all of the outer surface of one or more components of the prosthetic heart valve can be partially or fully coated with the chromium nitride (CrN) coating. The enhancement coating can be used to improve hardness, improve toughness, reduced friction, resistant impact wear, improve resistance to corrosion and oxidation, and/or form a reduced stick surface when in contact with many different materials. In accordance with one non-limiting embodiment, the chromium nitride (CrN) coating generally includes 40-85 wt. % Cr (and all values and ranges therebetween), 15-60 wt. % N (and all values and ranges therebetween), 0-10 wt. % Re (and all values and ranges therebetween), 0-10 wt. % Si (and all values and ranges therebetween), 0-2 wt. % O (and all values and ranges therebetween), and 0-2 wt. % C (and all values and ranges therebetween). In one non-limiting coating process, all or a portion of the outer surface of one or more components of the prosthetic heart valve are initially coated with Cr metal. The Cr metal coating can be applied by PVD, CVD, ALD and PE-CVD in an inert environment. The coating thickness of Cr metal is 0.5-15 microns. Thereafter, the Cr metal coating is exposed to nitrogen gas and/or a nitrogen containing gas compound to cause the nitrogen to react with the Cr metal coating to form a layer of CrN on the outer surface of the Cr metal coating and/or the outer surface of the one or more components of the prosthetic heart valve. Particles of Cr metal can optionally be mixed with nitrogen gas and/or a nitrogen containing gas compound to facilitate in the formation of the CrN coating. When Cr metal particles are used, the initial Cr coating layer on the outer surface of one or more components of the prosthetic heart valve can optionally be eliminated. In another non-limiting embodiment, the enhancement coating composition generally includes 65-80 wt. % Cr, 15-30 wt. % N, 0-8 wt. % Re, 0-1 wt. % Si, 0-1 wt. % O, and 0-1 wt. % C.

In accordance with another and/or alternative non-limiting aspect of the present disclosure, one or more components of the prosthetic heart valve can be partially or fully coated with an enhancement coating composition that includes a diamond-Like Carbon (DLC) coating. A portion or all of the outer surface of one or more components of the prosthetic heart valve can be partially or fully coated with the diamond-Like Carbon (DLC) coating. The enhancement coating can be used to improve hardness, improve toughness, reduced friction, resistant impact wear, improve resistance to corrosion and oxidation, improve biocompatibility, and/or form a reduced stick surface when in contact with many different materials. In one non-limiting embodiment, the diamond-Like Carbon (DLC) coating generally includes 60-99.99 wt. % C (and all values and ranges therebetween), 0-2 wt. % N (and all values and ranges therebetween), 0-10 wt. % Re (and all values and ranges therebetween), 0-20 wt. % Si (and all values and ranges therebetween), and 0-2 wt. % O (and all values and ranges therebetween). The carbon coating can be applied by PVD, CVD, ALD and PE-CVD in an inert environment. The carbon layer can be applied by use of methane and/or acetylene gas; however, other or additional carbon sources can be used. The coating thickness of the carbon is 0.5-15 microns. In another non-limiting embodiment, all or a portion of the outer surface of one or more components of the prosthetic heart valve are coated with the enhancement coating composition that generally includes 90-99.99 wt. % C, 0-1 wt. % N, 0-8 wt. % Re, 0-1 wt. % Si, and 0-1 wt. % O.

In accordance with another and/or alternative non-limiting aspect of the present disclosure, one or more components of the prosthetic heart valve can be partially or fully coated with an enhancement coating composition that includes a titanium nitride (TiN) coating. A portion or all of the outer surface of the one or more components of the prosthetic heart valve can include the titanium nitride (TiN) coating. The enhancement coating can be used to improve hardness, improve toughness, improve resistance to corrosion and oxidation, reduced friction, and/or form a reduced stick surface when in contact with many different materials. In one non-limiting embodiment, all or a portion of the outer surface of the one or more components of the prosthetic heart valve are optionally initially coated with Ti metal. The Ti metal coating, when applied, can be applied by PVD, CVD, ALD and PE-CVD in an inert environment. The coating thickness of Ti metal is 0.05-15 microns (and all values and ranges therebetween). As can be appreciated, the initial Ti coating is optional. Thereafter, the Ti metal coating, when applied, is exposed to nitrogen gas and/or a nitrogen containing gas compound and optionally titanium particles to cause the nitrogen to react with the Ti metal coating and/or titanium metal particles to form a layer of TiN on the outer surface of the Ti metal coating and/or the outer surface of the one or more components of the prosthetic heart valve. If a titanium layer is not preapplied, the TiN coating can be formed by exposing the outer surface of one or more components of the prosthetic heart valve to titanium particles and nitrogen gas and/or a nitrogen containing gas compound. The coating thickness of the TiN coating is generally 0.1-15 microns (and all values and ranges therebetween), and typically 0.2-2 microns.

In accordance with another and/or alternative non-limiting aspect of the present disclosure, one or more components of the prosthetic heart valve can be partially or fully coated with an enhancement coating composition that includes a titanium oxynitride or titanium nitride oxide (TiNOx) coating. A portion or all of the outer surface of the one or more components of the prosthetic heart valve can include the titanium oxynitride or titanium nitride oxide (TiNOx) coating. The enhancement coating can be used to improve hardness, improve toughness, improve resistance to corrosion and oxidation, reduced friction, form a reduced stick surface when in contact with many different materials, and/or promote nitric oxide formation on the surface of the coating. In one non-limiting embodiment, all or a portion of the outer surface of the one or more components of the prosthetic heart valve are optionally initially coated with Ti metal. The Ti metal coating, when applied, can be applied by PVD, CVD, ALD and PE-CVD in an inert environment. The coating thickness of Ti metal is 0.05-15 microns (and all values and ranges therebetween). As can be appreciated, the initial Ti coating is optional. Thereafter, the Ti metal coating is exposed to titanium particles and a nitrogen and oxygen mixture that can include nitrogen gas, oxygen gas, a nitrogen containing gas compound and/or an oxygen containing gas compound to cause the nitrogen and oxygen to react with the Ti metal coating, if such coating is used, and/or with the Ti metal particles to form a layer of TiNOx on the outer surface of the Ti metal coating and/or the outer surface of the one or more components of the prosthetic heart valve. The ratio of the N to the O can be varied to control the about of O in the TiNOx coating. If a titanium layer is not preapplied, the TiNOx coating can be formed by exposing the outer surface of one or more components of the prosthetic heart valve to titanium particles and a nitrogen and oxygen source such as nitrogen gas, oxygen gas, a nitrogen containing gas compound and/or an oxygen containing gas compound. The ratio of N to O when forming the TiNOx coating is generally 1:10 to 10:1 (and all values and ranges therebetween). The coating thickness of the TiNOx coating is generally 0.1-15 microns (and all values and ranges therebetween), and typically 0.2-2 microns. In another non-limiting embodiment, a TiNOx coating is applied to a portion or all of the outer surface of the one or more components of the prosthetic heart valve, and the TiNOx coating is formed by a) exposing the outer surface of a portion of all of the one or more components of the prosthetic heart valve to Ti particles (PVD, CVD, ALD and PE-CVD process) and/or a Ti containing solution to form a Ti layer on a portion of all of the one or more components of the prosthetic heart valve, and wherein the thickness of the Ti coating is 0.05-5 microns, and b) exposing the Ti coating to a nitrogen and oxygen source such as nitrogen gas, oxygen gas, a nitrogen containing gas compound and/or an oxygen containing gas compound to form a TiNOx coating, and wherein ratio of N to O when forming the TiNOx coating is generally 1:10 to 10:1, and wherein the coating thickness of the TiNOx coating is 0.2-5 microns. In another non-limiting embodiment, a TiNOx coating is applied to a portion or all of the outer surface of the one or more components of the prosthetic heart valve, and the TiNOx coating is formed by exposing a portion or all of the outer surface of the one or more components of the prosthetic heart valve to Ti particles and a nitrogen and oxygen source such as nitrogen gas, oxygen gas, a nitrogen containing gas compound and/or an oxygen containing gas compound to form a TiNOx coating, and wherein ratio of N to O when forming the TiNOx coating is generally 1:10 to 10:1, and wherein the coating thickness of the TiNOx coating is 0.2-5 microns. In another non-limiting embodiment, the enhancement coating composition generally includes 20-85 wt. % Ti (and all values and ranges therebetween), 0.5-35 wt. % N (and all values and ranges therebetween), 0-10 wt. % Re (and all values and ranges therebetween), and 0.5-35 wt. % O (and all values and ranges therebetween). In another non-limiting embodiment, a coating of TiNOx was formed on one or more components of the prosthetic heart valve by reactive physical vapor deposition in a vacuum chamber. Depending on the oxygen-nitrogen ratio during vapor deposition, a coating deposit of TiNOx with defined composition and resistivity can be coated on the outer surface of the one or more components of the prosthetic heart valve.

In accordance with another and/or alternative non-limiting aspect of the present disclosure, one or more components of the prosthetic heart valve can be partially or fully coated with an enhancement coating composition that includes a zirconium nitride (ZrN) coating. The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the one or more components of the prosthetic heart valve is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to nitrogen gas and/or a nitrogen containing gas compound to cause the nitrogen to react with the Zn metal coating to form a layer of ZrN on the outer surface of the Zr metal coating and/or the outer surface of the one or more components of the prosthetic heart valve. Particles of Zr metal can optionally be mixed with nitrogen gas and/or a nitrogen containing gas compound to facilitate in the formation of the ZrN coating. When Zr metal particles are used, the initial Zr coating layer on the outer surface of one or more components of the prosthetic heart valve can optionally be eliminated. The ZrN coating has been found to produce a gold-colored enhancement coating color. In another non-limiting embodiment, the enhancement coating composition generally includes 35-90 wt. % Zr (and all values and ranges therebetween), 5-25 wt. % N (and all values and ranges therebetween), 0-10 wt. % Re (and all values and ranges therebetween), 0-20 wt. % Si (and all values and ranges therebetween), 0-2 wt. % O (and all values and ranges therebetween), and 0-2 wt. % C (and all values and ranges therebetween). In another non-limiting embodiment, the enhancement coating composition generally includes 80-90 wt. % Zr, 10-20 wt. % N, 0-8 wt. % Rc, 0-1 wt. % Si, 0-1 wt. % O, and 0-1 wt. % C.

In accordance with another and/or alternative non-limiting aspect of the present disclosure, one or more components of the prosthetic heart valve can be partially or fully coated with an enhancement coating composition that includes a zirconium oxide (ZrO₂) coating. The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the one or more components of the prosthetic heart valve is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to oxygen gas and/or oxygen containing gas compound to cause the oxygen to react with the Zn metal coating to form a layer of zirconium oxide (ZrO₂) on the outer surface of the Zr metal coating and/or the outer surface of the one or more components of the prosthetic heart valve. Particles of Zr metal can optionally be mixed with the oxygen gas and/or an oxygen containing gas compound to facilitate in the formation of the ZrO₂ coating. When Zr metal particles are used, the initial Zr coating layer on the outer surface of one or more components of the prosthetic heart valve can optionally be eliminated. The zirconium oxide (ZrO₂) coating has been found to produce a blue colored enhancement coating color. In another non-limiting embodiment, the enhancement coating composition generally includes 35-90 wt. % Zr (and all values and ranges therebetween), 10-35 wt. % O (and all values and ranges therebetween), 0-2 wt. % N (and all values and ranges therebetween), 0-10 wt. % Re (and all values and ranges therebetween), 0-20 wt. % Si (and all values and ranges therebetween), and 0-2 wt. % C (and all values and ranges therebetween). In another non-limiting embodiment, the enhancement coating composition generally includes 70-80 wt. % Zr, 20-30 wt. %, 0-1 wt. % N, 0-8 wt. % Re, 0-1 wt. % Si, and 0-1 wt. % C.

In accordance with another and/or alternative non-limiting aspect of the present disclosure, one or more components of the prosthetic heart valve can be partially or fully coated with an enhancement coating composition that includes both a zirconium oxide (ZrO₂) coating and a zirconium nitride coating (ZrN). The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the metal alloy is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to a) both oxygen gas and/or oxygen containing gas compound and also to nitrogen gas and/or nitrogen containing gas compound, b) nitrogen gas and/or nitrogen containing gas compound and then to oxygen gas and/or oxygen containing gas compound, or c) oxygen gas and/or oxygen gas containing compound and then to nitrogen gas and/or nitrogen gas containing compound. The coating composition of the zirconium oxide (ZrO₂) coating and the zirconium nitride coating (ZrN) are similar or the same as discussed above. As discussed above, Particles of Zr metal can optionally be mixed with the oxygen gas and/or an oxygen containing gas compound to facilitate in the formation of the ZrO₂ coating and the nitrogen gas and/or nitrogen gas containing compound to facilitate in the formation of the ZrN coating. When Zr metal particles are used, the initial Zr coating layer on the outer surface of one or more components of the prosthetic heart valve can optionally be eliminated.

In accordance with another and/or alternative non-limiting aspect of the present disclosure, one or more components of the prosthetic heart valve can be partially or fully coated with an enhancement coating composition that includes a zirconium oxycarbide (ZrOC) coating. The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the metal alloy is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to a) both to oxygen gas and/or an oxygen containing gas compound and to carbon and/or a carbon containing gas compound (e.g., methane and/or acetylene gas), b) carbon and/or a carbon containing gas compound and then to oxygen gas and/or an oxygen containing gas compound, or c) oxygen gas and/or oxygen containing gas compound and then to carbon and/or carbon containing gas compound. Particles of Zr metal can optionally be mixed with the oxygen gas and/or an oxygen containing gas compound and the carbon and/or carbon containing gas compound to facilitate in the formation of the zirconium oxycarbide (ZrOC) coating. When Zr metal particles are used, the initial Zr coating layer on the outer surface of one or more components of the prosthetic heart valve can optionally be eliminated. In another non-limiting embodiment, the enhancement coating composition generally includes 40-95 wt. % Zr (and all values and ranges therebetween), 5-25 wt. % O (and all values and ranges therebetween), and 10-40 wt. % C (and all values and ranges therebetween), 0-2 wt. % N (and all values and ranges therebetween), 0-10 wt. % Re (and all values and ranges therebetween), and 0-20 wt. % Si (and all values and ranges therebetween). In another non-limiting embodiment, the enhancement coating composition generally includes 40-65 wt. % Zr, 5-25 wt. % O, and 25-40 wt. % C, 0-1 wt. % N, 0-8 wt. % Rc, and 0-1 wt. % Si.

In accordance with another and/or alternative non-limiting aspect of the present disclosure, one or more components of the prosthetic heart valve can be partially or fully coated with an enhancement coating composition that includes a zirconium oxynitride (ZnNxOy) [e.g., cubic ZrN:O, cubic ZrO₂:N, tetragonal ZrO₂:N, and monoclinic ZrO₂:N phase coatings]. A portion or all of the outer surface of the one or more components of the prosthetic heart valve can include the zirconium oxynitride (ZnNxOy). The enhancement coating can be used to improve hardness, improve toughness, improve resistance to corrosion and oxidation, reduced friction, form a reduced stick surface when in contact with many different materials, and/or promote nitric oxide formation on the surface of the coating. In one non-limiting embodiment, all or a portion of the outer surface of the one or more components of the prosthetic heart valve are optionally initially coated with Zr metal. The Zr metal coating, when applied, can be applied by PVD, CVD, ALD and PE-CVD in an inert environment. The coating thickness of Zr metal is 0.05-15 microns (and all values and ranges therebetween). As can be appreciated, the initial Zr coating is optional. Thereafter, the Zr metal coating is exposed to zirconium particles and a nitrogen and oxygen mixture that can include nitrogen gas, oxygen gas, a nitrogen containing gas compound and/or an oxygen containing gas compound to cause the nitrogen and oxygen to react with the Zr metal coating, if such coating is used, and/or with the Zr metal particles to form a layer of ZnNxOy on the outer surface of the Zr metal coating and/or the outer surface of the one or more components of the prosthetic heart valve. The ratio of the N to the O can be varied to control the about of O and N in the ZrNxOy coating. If a zirconium layer is not preapplied, the ZrNxOy coating can be formed by exposing the outer surface of one or more components of the prosthetic heart valve to zirconium particles and a nitrogen and oxygen source such as nitrogen gas, oxygen gas, a nitrogen containing gas compound and/or an oxygen containing gas compound. The ratio of N to O when forming the ZrNxOy coating is generally 1:10 to 10:1 (and all values and ranges therebetween). The coating thickness of the ZrNxOy coating is generally 0.1-15 microns (and all values and ranges therebetween), and typically 0.2-2 microns. In another non-limiting embodiment, a ZrNxOy coating is applied to a portion or all of the outer surface of the one or more components of the prosthetic heart valve, and the ZrNxOy coating is formed by a) exposing the outer surface of a portion of all of the one or more components of the prosthetic heart valve to Zr particles (PVD, CVD, ALD and PE-CVD process) and/or a Zr containing solution to form a Zr layer on a portion of all of the one or more components of the prosthetic heart valve, and wherein the thickness of the Zr coating is 0.05-5 microns, and b) exposing the Zr coating to a nitrogen and oxygen source such as nitrogen gas, oxygen gas, a nitrogen containing gas compound and/or an oxygen containing gas compound to form a ZrNxOy coating, and wherein ratio of N to O when forming the ZrNxOy coating is generally 1:10 to 10:1, and wherein the coating thickness of the ZrNxOy coating is 0.2-5 microns. In another non-limiting embodiment, a ZrNxOy coating is applied to a portion or all of the outer surface of the one or more components of the prosthetic heart valve, and the ZrNxOy coating is formed by exposing a portion or all of the outer surface of the one or more components of the prosthetic heart valve to Zr particles and a nitrogen and oxygen source such as nitrogen gas, oxygen gas, a nitrogen containing gas compound and/or an oxygen containing gas compound to form a ZrNxOy coating, and wherein ratio of N to O when forming the ZrNxOy coating is generally 1:10 to 10:1, and wherein the coating thickness of the ZrNxOy coating is 0.2-5 microns. In another non-limiting embodiment, the enhancement coating composition generally includes 20-85 wt. % Zr (and all values and ranges therebetween), 0.5-35 wt. % N (and all values and ranges therebetween), and 0.5-35 wt. % O (and all values and ranges therebetween). In another non-limiting embodiment, a coating of ZrNxOy was formed on one or more components of the prosthetic heart valve by reactive physical vapor deposition in a vacuum chamber. Depending on the oxygen-nitrogen ratio during vapor deposition, a coating deposit of ZrNxOy with defined composition and resistivity can be coated on the outer surface of the one or more components of the prosthetic heart valve.

In accordance with another and/or alternative non-limiting aspect of the present disclosure, one or more components of the prosthetic heart valve can be partially or fully coated with an enhancement coating composition that includes a zirconium-nitrogen-carbon (ZrNC) coating. The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the one or more components of the prosthetic heart valve is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to nitrogen gas and/or a nitrogen containing gas compound and then to carbon and/or a carbon containing gas compound (e.g., methane and/or acetylene gas). The color of the ZINC will vary depending on the amount of C and N in the coating. Particles of Zr metal can optionally be mixed with nitrogen gas and/or a nitrogen containing gas compound and the carbon and/or a carbon containing gas compound to facilitate in the formation of the ZrNC coating. When Zr metal particles are used, the initial Zr coating layer on the outer surface of one or more components of the prosthetic heart valve can optionally be eliminated. In one non-limiting embodiment, the enhancement coating composition generally includes 40-95 wt. % Zr (and all values and ranges therebetween), 5-40 wt. % N (and all values and ranges therebetween), and 5-40 wt. % C (and all values and ranges therebetween), 0-2 wt. % O (and all values and ranges therebetween), 0-10 wt. % Re (and all values and ranges therebetween), and 0-20 wt. % Si (and all values and ranges therebetween). In another non-limiting embodiment, the enhancement coating composition generally includes 40-80 wt. % Zr, 5-25 wt. % N, and 5-25 wt. % C, 0-1 wt. % 0, 0-8 wt. % Re, and 0-1 wt. % Si.

In accordance with another and/or alternative non-limiting aspect of the present disclosure, one or more components of the prosthetic heart valve (e.g., frame, inner skirt, outer skirt, leaflets, material used to secure leaflets to frame, etc.) can optionally be partially (e.g., 1% to 99.99% and all values and ranges therebetween) or fully be coated with one or more agents. When one or more agents are coated on one or more components of the prosthetic heart valve, and one or more components of the prosthetic heart valve includes an enhancement coating, such one or more agents are generally coated on the outer surface of the enhancement coating. If one or more components of the prosthetic heart valve are absent an enhancement coating, such one or more agents can be directly applied to the outer surface of the one or more components of the prosthetic heart valve are absent an enhancement coating. The term “agent” includes, but is not limited to a substance, pharmaceutical, biologic, veterinary product, drug, and analogs or derivatives otherwise formulated and/or designed to prevent, inhibit and/or treat one or more clinical and/or biological events, and/or to promote healing. Non-limiting examples of clinical events that can be addressed by one or more agents include, but are not limited to, viral, fungus and/or bacterial infection; vascular diseases and/or disorders; lymphatic diseases and/or disorders; cancer; implant rejection; pain; nausea; swelling; organ failure; immunity diseases and/or disorders; cell growth inhibitors, blood diseases and/or disorders; heart diseases and/or disorders; neuralgia diseases and/or disorders; fatigue; genetic diseases and/or disorders; trauma; cramps; muscle spasms; tissue repair; nerve repair; neural regeneration and/or the like.

In accordance with another and/or alternative non-limiting aspect of the present disclosure, one or more portion of the prosthetic heart valve (e.g., frame, etc.) can optionally include a marker material that facilitates enabling the prosthetic heart valve to be properly positioned in the treatment area of the heart. The marker material is typically designed to be visible to electromagnetic waves (e.g., x-rays, microwaves, visible light, infrared waves, ultraviolet waves, etc.); sound waves (e.g., ultrasound waves, etc.); magnetic waves (e.g., MRI, etc.); and/or other types of electromagnetic waves (e.g., microwaves, visible light, infrared waves, ultraviolet waves, etc.).

In accordance with another and/or alternative non-limiting aspect of the present disclosure, one or more portions of the prosthetic heart valve can optionally include one or more surface structures (e.g., pore, channel, pit, rib, slot, notch, bump, teeth, needle, well, hole, groove, etc.). These structures can be at least partially formed by MEMS (e.g., micro-machining, etc.) technology and/or other types of technology (e.g., 3D printing, etc.).

In accordance with another and/or alternative non-limiting aspect of the present disclosure, one or more portions of the prosthetic heart valve (e.g., frame, leaflets, inner skirt, outer skirt, etc.) can optionally include one or more micro-structures (e.g., micro-needle, micro-pore, micro-cylinder, micro-cone, micro-pyramid, micro-tube, micro-parallelopiped, micro-prism, micro-hemisphere, teeth, rib, ridge, ratchet, hinge, zipper, zip-tie-like structure, etc.) on the surface of the one or more portions of the prosthetic heart valve. As defined herein, a “micro-structure” is a structure having at least one dimension (e.g., average width, average diameter, average height, average length, average depth, etc.) that is no more than about 2 mm, and typically no more than about 1 mm. Non-limiting examples of structures that can be formed on the one or more portions of the prosthetic heart valve are illustrated in U.S. Pat. Nos. 7,255,710 and 7,141,063, which are incorporated herein by reference.

In accordance with another and/or alternative non-limiting aspect of the present disclosure, the frame of the prosthetic heart valve can optionally be an expandable device that can be expanded by use of some other device (e.g., balloon, etc.).

In accordance with another and/or alternative non-limiting aspect of the present disclosure, the frame of the prosthetic heart valve can optionally be fabricated from a material having no or substantially no shape-memory characteristics.

In accordance with another and/or alternative non-limiting aspect of the present disclosure, the frame of the prosthetic heart valve can optionally be fabricated from a material having shape-memory characteristics.

In accordance with another and/or alternative aspect of the present disclosure, the metal alloy used to at least partially form the frame of the prosthetic heart valve is initially formed into a blank, a rod, a tube, etc., and then finished into final form by one or more finishing processes. The metal alloy blank, rod, tube, etc., can be formed by various techniques such as, but not limited to, 1) melting the metal alloy and/or metals that form the metal alloy (e.g., vacuum arc melting, etc.) and then extruding and/or casting the metal alloy into a blank, rod, tube, etc., 2) melting the metal alloy and/or metals that form the metal alloy, forming a metal strip, and then rolling and welding the strip into a blank, rod, tube, etc., 3) consolidating the metal powder of the metal alloy and/or metal powder of metals that form the metal alloy into a blank, rod, tube, etc. (e.g., pressure and/or sintering process), or 4) 3-D printing the metal powder of the metal alloy and/or metal powder of metals that form the metal alloy into a blank, rod, tube, etc.

In accordance with another and/or alternative non-limiting aspect of the present disclosure, there is provided a prosthetic heart valve that is configured to be inserted into a portion of a heart (e.g., the aortic valve, tricuspid valve, pulmonary valve, mitral valve). The frame of the prosthetic heart valve can be at least partially formed of a plastically-expandable material that permits crimping of the frame to a smaller profile for delivery and expansion of the prosthetic heart valve to a larger profile. The expansion of the crimped frame can be optionally be by use of an expansion device such as, but not limited to, a balloon of on a balloon catheter. As can be appreciated, the frame of the prosthetic heart valve can be formed of a self-expanding metal alloy.

In accordance with another and/or alternative non-limiting aspect of the present disclosure, when the frame is partially or fully formed of a refractory metal alloy or a metal alloy that includes at 5 awt. % (e.g., 5-99 awt. % and all values and ranges therebetween) rhenium, the use of such metal alloy can result in one or more advantages over frames of prosthetic heart valves formed from other materials. These one or more advantages include, but are not limited to:
The metal alloy can have increased strength and/or hardness as compared with stainless steel, chromium-cobalt alloys, or titanium alloys, thus a less quantity of metal alloy can be used in the frame of the prosthetic heart valve to achieve similar strengths as compared to frames of the prosthetic heart valve formed of different metals. As such, the resulting frame of the prosthetic heart valve can be made smaller, include smaller cross-sectional struts and/or posts, and be less bulky without sacrificing the strength and durability of the frame of the prosthetic heart valve. The frame of the prosthetic heart valve can also have a smaller profile, thus can be inserted into smaller areas, openings, and/or passageways.
The increased strength of the metal alloy can result in the increased radial strength of the frame of the prosthetic heart valve. For instance, the thickness and/or cross-sectional area of the wall, struts and/or posts of the frame of the prosthetic heart valve can be made thinner and achieve a similar or improved radial strength as compared with frames having thicker walls, struts and/or posts that are formed of stainless steel, cobalt and chromium alloy, or titanium alloy.
The metal alloy used to form the frame of the prosthetic heart valve can have improved stress-strain properties, bendability properties, elongation properties, and/or flexibility properties as compared to similar sized and shaped frames that are formed of stainless steel and chromium-cobalt alloys.
The metal alloy can have a reduced degree of recoil during the crimping and/or expansion of the frame of the prosthetic heart valve as compared to similar sized and shaped frames that are formed of stainless steel, chromium-cobalt alloys, or titanium alloys. The frame of the prosthetic heart valve formed of the metal alloy better maintains its crimped form and/or better maintains its expanded form after expansion due to the use of the metal alloy. As such, when the frame of the prosthetic heart valve is to be mounted onto a delivery device when the frame of the prosthetic heart valve is crimped, the frame of the prosthetic heart valve better maintains its crimped profile and shape after each crimping process as compared to similar sized and shaped frames that are formed of stainless steel, chromium-cobalt alloys, or titanium alloys. Also, the frame of the prosthetic heart valve better maintains its expanded profile after expansion to facilitate in the success of the prosthetic heart valve in the treatment area as compared to similar sized and shaped frames that are formed of stainless steel, chromium-cobalt alloys, or titanium alloys.
The metal alloy used to partially or fully form the frame of the prosthetic heart valve can result in the frame of the prosthetic heart valve better conforming to an irregularly shaped body passageway when expanded in the treatment area as compared to similar sized and shaped frames that are formed of stainless steel, chromium-cobalt alloys, or titanium alloys.
The metal alloy used to partially or fully form the frame of the prosthetic heart valve can have improved radiopaque properties as compared to similar sized and shaped frames that are formed of stainless steel, chromium-cobalt alloys, or titanium alloys.
The metal alloy used to partially or fully form the frame of the prosthetic heart valve can have improved fatigue ductility when subjected to cold-working as compared to similar sized and shaped frames that are formed of stainless steel, chromium-cobalt alloys, or titanium alloys.
The metal alloy used to partially or fully form the frame of the prosthetic heart valve can have improved durability as compared to similar sized and shaped frames that are formed of stainless steel, chromium-cobalt alloys, or titanium alloys.
The metal alloy used to partially or fully form the frame of the prosthetic heart valve can have improved hydrophilicity as compared to similar sized and shaped frames that are formed of stainless steel, chromium-cobalt alloys, or titanium alloys.
The metal alloy used to partially or fully form the frame of the prosthetic heart valve can have reduced ion release in the body passageway as compared to similar sized and shaped frames that are formed of stainless steel, chromium-cobalt alloys, or titanium alloys.
The metal alloy used to partially or fully form the frame of the prosthetic heart valve can be less of an irritant to the cells about the implanted frame as compared to similar sized and shaped frames that are formed of stainless steel, chromium-cobalt alloys, or titanium alloys, thus can result in reduced inflammation, faster healing, increased success rates of the medical device, and/or slower growth rate of cells from the heart onto the frame and/or leaflets of the prosthetic heart valve. For instance, when the frame of the prosthetic heart valve is expanded in the heart, some minor damage to the region about the expanded frame can occur. When the body begins to heal such minor damage, the body can have a less adverse reaction to the presence of the metal alloy compared to other metals such as stainless steel, cobalt-chromium alloy, or titanium alloy. When one or more portion of the prosthetic heart valve includes an enhancement coating (e.g., TiNOx, ZrNxOy, etc.), such coating can be used to further reduce irritation to the cells about the expanded prosthetic heart valve and/or facilitate in the generation of nitric oxide on the surface of the coating, and thus reduce and/or slow the growth rate of cells from the heart onto the frame and/or leaflets of the prosthetic heart valve.
The metal alloy used to partially or fully form the frame can have a magnetic susceptibility that is lower than similar sized and shaped frames that are formed of stainless steel, chromium-cobalt alloys, or titanium alloys, thus resulting in less incidence of potential defects to the prosthetic heat valve medical device or complications to the patent after implantation of the prosthetic heart valve when the patient is subjected to an MRI or other medical device that generates a strong magnetic field.

One non-limiting object of the present disclosure is the provision of a prosthetic heart valve wherein a portion or all of the outer surface of one or more components (e.g., frame, one or more leaflets, inner skirt, outer skirt, material used to connect leaflets to frame, etc.) of the prosthetic heart valve are coated with an enhancement coating.

Another and/or alternative non-limiting object of the present disclosure is the provision of a prosthetic heart valve wherein a portion or all of the outer surface of one or more components of the prosthetic heart valve are coated with an enhancement coating that includes TiNOx and/or ZrNxOy so as to promote the formation of nitric oxide on the surface of the coating.

Another and/or alternative non-limiting object of the present disclosure is the provision of a prosthetic heart valve wherein a portion or all of the frame of the prosthetic heart valve includes a refractory metal alloy or a metal alloy that includes at least 5 awt. % (e.g., 5-99 awt. % and all values and ranges therebetween) rhenium.

Another and/or alternative non-limiting object of the present disclosure is the provision of a prosthetic heart valve that has improved procedural success rates.

Another and/or alternative non-limiting object of the present disclosure is the provision of a prosthetic heart valve that reduces the rate at which heart cells grown onto one or more components of the prosthetic heart valve after implantation of the prosthetic heart valve.

Another and/or alternative non-limiting object of the present disclosure is the provision of a prosthetic heart valve that reduces the rate at which heart cells grow onto one or more components of the prosthetic heart valve after implantation of the prosthetic heart valve without the use of a coating of one or more agents on the outer surface of the components of the prosthetic heart valve. Traditionally, cell growth rate onto the prosthetic heart valve, when control was attempted, was accomplished by the use of drug coatings on the prosthetic heart valve. The time period of effectiveness when using such drug coatings was limited due to the drug coating eventually fully releasing from the prosthetic heart valve over time. Also, the use of drug coatings to inhibit cells growth about the implanted prosthetic heart valve can result in premature failure of the prosthetic heart valve due to undesired damage to the tissue about the implanted prosthetic heart valve, inhibition of cells about the frame to grow and secure the frame in position in the treatment area, and inadvertent promotion of restenosis in the treatment area. The use of the enhancement coating on a portion of the full the outer surface of one or more components of the prosthetic heart valve can result is less irritation and/or bioincompatibility in the treatment area after implantation of the prosthetic heart valve, thus reducing the rate at which heart cells grow onto one or more components of the prosthetic heart valve after implantation of the prosthetic heart valve.

Another and/or alternative non-limiting object of the present disclosure is the provision of a prosthetic heart valve that is coated with an enhancement coating used to a) reduced metal ion release of the metal material from the frame of the prosthetic heart valve, b) reduce the rate of corrosion on the metal that forms the frame of the prosthetic heart valve and/or c) reduces the rate of structural valve disease (SVD) by i) reducing neointimal hyperplasia/cell overgrowth onto one or more portions of the prosthetic heart valve after implantation in the treatment area, ii) reducing infection about the prosthetic heart valve after implantation in the treatment area, iii) reducing platelet activation about the prosthetic heart valve after implantation in the treatment area, iv) reducing thrombosis about the prosthetic heart valve after implantation in the treatment area, v) reducing restenosis about the prosthetic heart valve after implantation in the treatment area, vi) reducing the incidence of nickel from the frame of the prosthetic heart valve reacting with cells about the prosthetic heart valve after implantation in the treatment area, vii) reducing inflammatory cell response about the prosthetic heart valve after implantation in the treatment area, viii) promoting endothelial cell angiogenesis about the prosthetic heart valve after implantation in the treatment area.

Another and/or alternative non-limiting object of the present disclosure is the provision of a prosthetic heart valve that is coated with an enhancement coating (e.g., metal oxynitride layer) that facilitates in the formation of a) nitric oxide (NO) production, b) stimulation of endothelial cells, and/or c) a modulation of endothelial cells.

Another and/or alternative non-limiting object of the present disclosure is the provision of a prosthetic heart valve that includes a plurality of components; two of the components include a metallic frame and at least one leaflet; the metallic frame is directly or indirectly attached to the at least one leaflet; the at least one leaflet is configured to at least partially control blood flow through the metallic frame; an outer surface of at least one component of the prosthetic heart valve includes an enhancement material that i) provides nitric oxide or its precursors nitrogen and oxygen, ii) promotes generation of nitric oxide in adjacent tissue, and/or iii) promotes transport of nitric oxide to adjacent tissue.

Another and/or alternative non-limiting object of the present disclosure is the provision of a prosthetic heart valve wherein the enhancement material includes an outer metal oxynitride layer.

Another and/or alternative non-limiting object of the present disclosure is the provision of a prosthetic heart valve wherein the outer metal oxynitride layer includes titanium oxynitride and/or zirconium oxynitride.

Another and/or alternative non-limiting object of the present disclosure is the provision of a prosthetic heart valve wherein the outer metal oxynitride layer has a thickness of at least 10 nanometers.

Another and/or alternative non-limiting object of the present disclosure is the provision of a prosthetic heart valve wherein the outer metal oxynitride layer has an oxygen to nitrogen atomic ratio of 1:10 to 10:1.

Another and/or alternative non-limiting object of the present disclosure is the provision of a prosthetic heart valve wherein the enhancement coating includes the outer metal oxynitride layer and a metallic adhesion layer; the outer metal oxynitride layer is coated on an outer surface of the metallic adhesion layer; the metallic adhesion layer is coated on an outer surface of the metallic frame.

Another and/or alternative non-limiting object of the present disclosure is the provision of a prosthetic heart valve wherein the metallic adhesion layer includes titanium metal or zirconium metal.

Another and/or alternative non-limiting object of the present disclosure is the provision of a prosthetic heart valve wherein the metallic adhesion layer has a thickness of at least 1 nanometer.

Another and/or alternative non-limiting object of the present disclosure is the provision of a prosthetic heart valve wherein the enhancement coating includes no more than 0.1 wt. % nickel and/or no more than 0.1 wt. % cobalt.

Another and/or alternative non-limiting object of the present disclosure is the provision of a prosthetic heart valve wherein the metallic frame includes no more than 0.1 wt. % nickel and/or no more than 0.1 wt. % cobalt.

Another and/or alternative non-limiting object of the present disclosure is the provision of a prosthetic heart valve wherein the enhancement coating is only coated on an outer surface of at least a portion of the metallic frame.

Another and/or alternative non-limiting object of the present disclosure is the provision of a prosthetic heart valve wherein the enhancement coating is coated on at least a portion of an outer surface of the metallic frame and the at least one leaflet.

Another and/or alternative non-limiting object of the present disclosure is the provision of a prosthetic heart valve wherein the enhancement coating is coated on at least a portion of an outer surface of the metallic frame, the at least one leaflet, the inner skirt and the outer skirt.

Another and/or alternative non-limiting object of the present disclosure is the provision of a prosthetic heart valve wherein the metallic frame is configured to foreshorten 0-5% of a longitudinal length of the metallic frame when the metallic frame is expanded from a crimped state to an expanded state.

Another and/or alternative non-limiting object of the present disclosure is the provision of a method for repairing a heart valve; the method comprising a) providing a prosthetic heart valve that is crimped about a delivery system; the prosthetic heart valve includes a plurality of components; two of the components include a metallic frame and at least one leaflet; the metallic frame is directly or indirectly attached to the at least one leaflet; the at least one leaflet is configured to at least partially control blood flow through the metallic frame; an outer surface of at least one component of the prosthetic heart valve includes an enhancement material that i) provides nitric oxide or its precursors nitrogen and oxygen, ii) promotes generation of nitric oxide in adjacent tissue, iii) promotes transport of nitric oxide to adjacent tissue, and/or iv) generates nitric oxide (NO) via nitric oxide donation in a local environment of the prosthetic heart valve; b) positioning the prosthetic heart valve in a treatment area of a heart; and, c) expanding the metallic frame from a crimped state to an expanded state while the prosthetic heart valve is in the treatment area of the heart.

Another and/or alternative non-limiting object of the present disclosure is the provision of a method for repairing a heart valve wherein the metallic frame has no more than 5% recoil after the metallic frame has been expanded from the crimped state to the expanded state.

Another and/or alternative non-limiting object of the present disclosure is the provision of a method for repairing a heart valve the nitric oxide donation includes use of a nitric oxide donating compound; the nitric oxide donating compound is a) a direct nitric oxide donator, wherein the direct nitric oxide donator includes S—NO—N-acetyl-L-cysteine, Molsidomine, Diethylamino-NONOate, Spermine NONOate, S—NO-Glutathione, and/or S—NO-diclofenac, b) a metabolic nitric oxide donator, wherein the metabolic nitric oxide donator includes nitroglycerin, amyl nitrite, isosorbide dinitrate, isosorbide mononitrate, and/or nicorandil, and/or c) a bifunctional nitric oxide donator, wherein the bifunctional nitric oxide donator includes nitroaspirins and/or S-Nitroso-NTHEs.

Another and/or alternative non-limiting object of the present disclosure is the provision of a method for repairing a heart valve wherein at least one of the leaflets is formed of a biological tissue material, and wherein the nitric oxide donating compound is a) adhered to and/or permeated within interstices of the biological tissue material, b) chemically bound to an extracellular matrix of the biological tissue material, and/or c) chemically bound to free amine residues on collagen of the biological tissue material via crosslinking.

Another and/or alternative non-limiting object of the present disclosure is the provision of a method for repairing a heart valve wherein the crosslinking is at least partially achieved by use of one or more of glutaraldehyde, formaldehyde, genipin, carbodiimides, dialdehyde starch, temperature, and/or UV light crosslinking.

Another and/or alternative non-limiting object of the present disclosure is the provision of a method for repairing a heart valve wherein the crosslinking is reduced via a reducing agent to inhibit or prevent reversibility of the cross-linking.

Another and/or alternative non-limiting object of the present disclosure is the provision of a method for repairing a heart valve wherein the prosthetic heart valve includes biological tissue material with a nitric oxide donating compound that is chemically bound to a secondary structure acting as an intermediary between the nitric oxide donating compound and collagen and/or the nitric oxide donating compound and a crosslinking agent.

Another and/or alternative non-limiting object of the present disclosure is the provision of a method for repairing a heart valve wherein the secondary structure possesses residues congruent with crosslinking of tissue-based collagen structures; the residues include aldehyde residues, carboxyl residues, and/or amine residues.

Another and/or alternative non-limiting object of the present disclosure is the provision of a method for repairing a heart valve wherein the nitric oxide donating compound is embedded within the interstices of the biological tissue material.

Another and/or alternative non-limiting object of the present disclosure is the provision of a method for repairing a heart valve wherein the embedded nitric oxide donating compound that is retained within the biological tissue material is configured to release nitric oxide into the local environment.

Another and/or alternative non-limiting object of the present disclosure is the provision of a method for repairing a heart valve wherein the embedded nitric oxide donating compound itself is released into the local environment.

Another and/or alternative non-limiting object of the present disclosure is the provision of a method for repairing a heart valve wherein the nitric oxide donating compound is introduced into interstices of the biological tissue material via serial immersion into a treatment solution then drying the treated biological tissue material.

Another and/or alternative non-limiting object of the present disclosure is the provision of a method for repairing a heart valve wherein the treatment solution includes the nitric oxide donor compound that is within a dimensional stabilizer compound that enables the treated biological tissue material be stable in standard air composition.

Another and/or alternative non-limiting object of the present disclosure is the provision of a method for repairing a heart valve wherein the dimensional stabilizer compound includes a polyol compound.

Another and/or alternative non-limiting object of the present disclosure is the provision of a method for repairing a heart valve wherein the polyol compound includes ethylene glycol, propylene glycol, and/or glycerol.

Another and/or alternative non-limiting object of the present disclosure is the provision of a method for repairing a heart valve wherein the biological tissue material is treated with a dimensional stabilizer compound at a time that is concurrent with or subsequent to treatment adherence of the nitric oxide donor compound.

Another and/or alternative non-limiting object of the present disclosure is the provision of a method for repairing a heart valve wherein the secondary structures include a polymeric material with nitric oxide generating compound that is adhered to or is permeated within pores of the polymeric material.

Another and/or alternative non-limiting object of the present disclosure is the provision of a method for repairing a heart valve wherein the polymetric material includes polyethers, polyesters, polyurethanes, and/or polycarbons.

Another and/or alternative non-limiting object of the present disclosure is the provision of a method for repairing a heart valve wherein the polymetric material includes one or more compositional elements; the compositional elements include macrodiol segments, polyol segments, and/or cyanates.

Another and/or alternative non-limiting object of the present disclosure is the provision of a prosthetic heart valve that includes a plurality of components; two of the components include a metallic frame and at least one leaflet; the metallic frame is directly or indirectly attached to the at least one leaflet; the at least one leaflet is configured to at least partially control blood flow through the metallic frame; an outer surface of at least one component of the prosthetic heart valve includes an enhancement material; the enhancement material is formulated to i) provide nitric oxide, and/or ii) promote generation of nitric oxide; the enhancement material is at least partially formulated of oxynitride, and wherein the enhancement material optionally includes metal oxynitride, and wherein the metal oxynitride optionally includes titanium oxynitride and/or zirconium oxynitride, and wherein the metal oxynitride optionally has a thickness of at least 10 nanometers, and wherein the metal oxynitride optionally has an oxygen to nitrogen atomic ratio of 1:10 to 10:1, and wherein the enhancement coating optionally is at least partially coated on a metallic adhesion layer; the metal oxynitride layer is optionally coated on an outer surface of the metallic adhesion layer or the metallic adhesion layer is optionally coated on an outer surface of the metallic frame, and wherein the metallic adhesion layer optionally is includes titanium metal or zirconium metal, and wherein the metallic adhesion layer optionally has a thickness of at least 1 nanometers, and wherein the plurality of components optionally further include one or more of an inner skirt, an outer skirt, and/or sutures, and wherein the enhancement coating optionally includes no more than 0.1 wt. % nickel and/or no more than 0.1 wt. % cobalt, and wherein the adhesion layer optionally includes no more than 0.1 wt. % nickel and/or no more than 0.1 wt. % cobalt, and wherein the metallic frame optionally includes no more than 0.1 wt. % nickel and/or no more than 0.1 wt. % cobalt, and wherein the enhancement coating is optionally only coated on or over an outer surface of at least a portion of the metallic frame, and wherein the enhancement coating is optionally coated on or over at least a portion of an outer surface of the metallic frame, and at least one leaflet, the inner skirt and/or the outer skirt, and wherein the metallic frame is optionally configured to foreshorten 0-5% of a longitudinal length of the metallic frame when the metallic frame is expanded from a crimped state to an expanded state, and wherein the metallic frame is optionally formed of a) standard stainless steel, b) standard cobalt-chromium alloy, c) standard titanium-aluminum-vanadium alloy, d) standard aluminum alloy, e) standard nickel alloy, f) standard titanium alloy, g) standard tungsten alloy, h) standard molybdenum alloy, i) standard copper alloy, j) standard beryllium-copper alloy, k) standard titanium-nickel alloy, 1) refractory metal alloy, or m) metal alloy that includes at least 5 atomic weight percent (awt. %) rhenium.

These and other advantages will become apparent to those skilled in the art upon the reading and following of this description.

### BRIEF DESCRIPTION OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. Reference may now be made to the drawings, which illustrate various embodiments that the disclosure may take in physical form and in certain parts and arrangement of parts wherein:

FIG. 1A is an illustration of a TAV in accordance with the present disclosure.

FIG. 1B is a portion of a prior art catheter.

FIGS. 1C-1E illustrate a typical TAVR procedure for inserting the TAV into a valve of a heart.

FIG. 2 is an illustration of the TAV of FIG. 1A illustrating features of the struts and posts of the frame.

FIG. 3 is a front elevation view of another non-limiting frame in the expanded state that can be used as a frame for a prosthetic heart valve.

FIG. 4 is a front view of the flat frame of FIG. 3.

FIG. 5 is a front view of a flat frame of another non-limiting frame in the expanded state that can be used as a frame for a prosthetic heart valve.

FIG. 6 is a cross-sectional view of a section of a frame that illustrates an enhancement coating on the outer surface of the section of the frame.

### DESCRIPTION OF NON-LIMITING EMBODIMENTS OF THE DISCLOSURE

A more complete understanding of the articles/devices, processes and components disclosed herein can be obtained by reference to the accompanying drawings. These figures are merely schematic representations based on convenience and the case of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

For the sake of simplicity, the attached figures may not show the various ways (readily discernable, based on this disclosure, by one of ordinary skill in the art) in which the disclosed system, method and apparatus can be used in combination with other systems, methods and apparatuses. Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

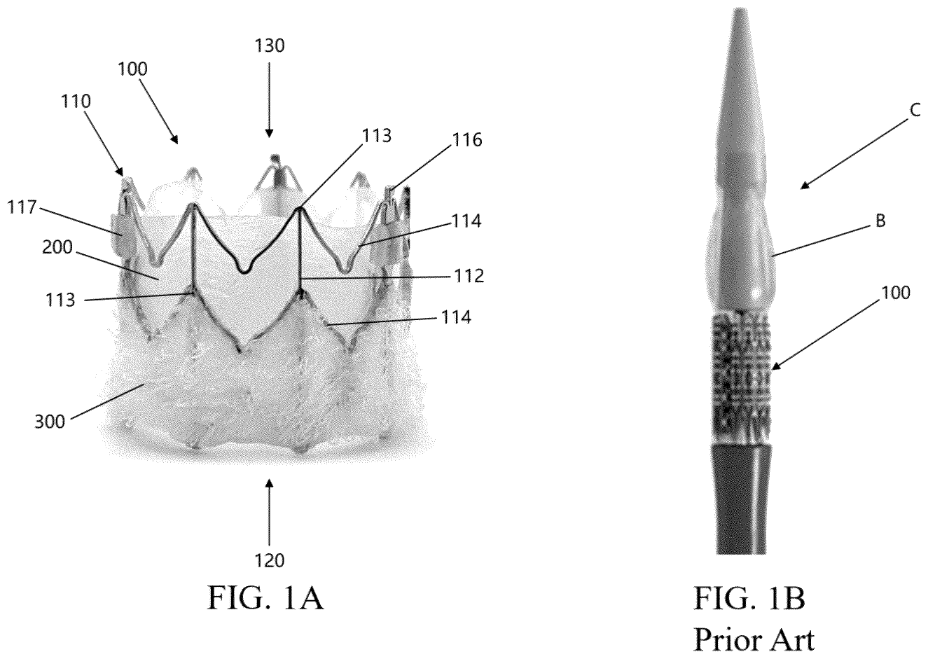

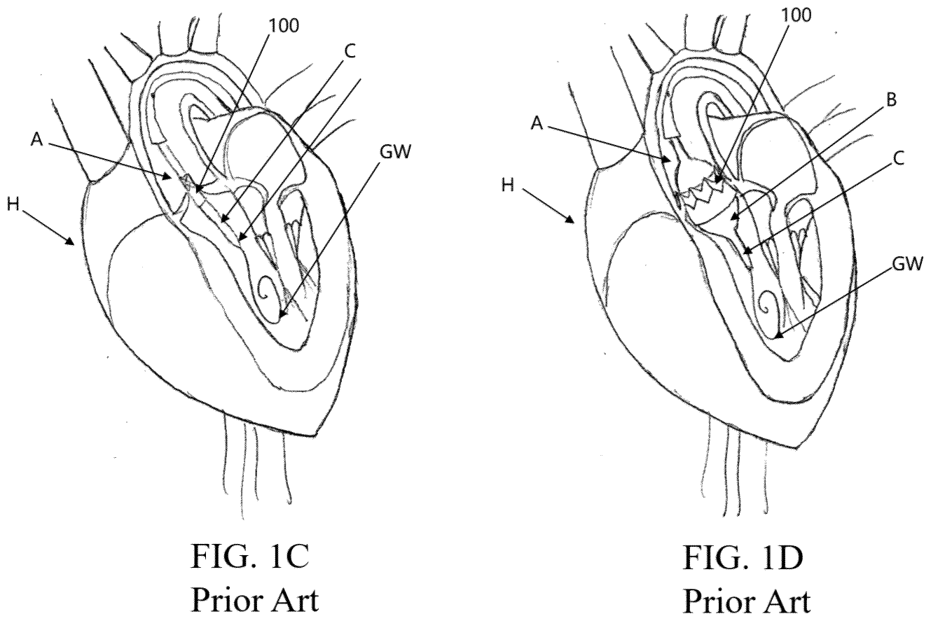

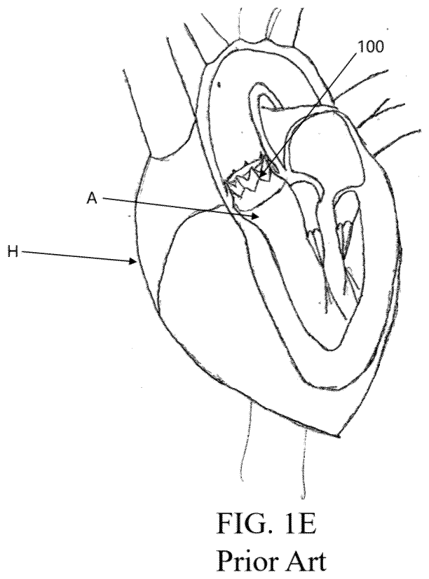

Referring now to FIGS. 1A-1B, there are illustrations of an implantable prosthetic heart valve 100 (e.g., TAV) that can be used for insertion in a valve region (e.g., aortic valve, etc.) of a heart. The prosthetic heart valve 100 can be implanted in the annulus of the native aortic valve; however, the prosthetic heart valve 100 also can be configured to be implanted in other valves of the heart.

Referring now to FIG. 1A, the prosthetic heart valve 100 generally comprises a frame 110 formed of a plurality of posts and/or struts 112, 114, a plurality of strut joints 113, a leaflet structure 200 that is supported by the frame 110, and an inner skirt 300 that is secured to the outer surface of the frame 110 and/or leaflet structure 200. The prosthetic heart valve 100 has a “lower” end 120 and an “upper” end 130, wherein the lower end 120 of the prosthetic heart valve 100 is the inflow end and the upper end 130 of the prosthetic heart valve 100 is the outflow end when inserted into a treatment site.

The configuration of the frame 110 of the prosthetic heart valve 100 is non-limiting. Many different frame configurations can be used for the frame 110 of the prosthetic heart valve 100. The frame 110 includes a plurality of spaced, vertically extending struts or posts 112, or non-vertically extending struts 114 that are connected together at strut joints 113. As can be appreciated, the frame 110 can be fully formed of non-vertically extending struts 114 that are connected together at strut joints 113.

As illustrated in FIG. 1A, the frame 110 has a plurality of vertically extending struts or posts 112 that are position about the upper portion of the frame 110. The vertically extending struts or posts 112 are interconnected via a lower row of circumferentially non-vertically extending struts 114 at strut joints 113 and an upper row of circumferentially non-vertically extending struts 114. The non-vertically extending struts 114 can be arrangement in a variety of patterns (e.g., zig-zag pattern, saw-tooth pattern, triangular pattern, polygonal pattern, oval pattern, S-shaped, Y-shaped, H-shaped, E-shaped, V-shaped, Z-shaped, L-shaped, J-sped, W-shaped, U-shaped, N-shaped, M-shaped, C-shaped, X-shaped, F-shaped, etc.). One or more of the posts and/or struts 112, 114 can have the same or different a) thicknesses, b) cross-sectional shape, and/or c) cross-sectional area along a portion or all of the longitudinal length.

The frame 110 is partially or fully formed of a metal material. Non-limiting metal materials include a) standard stainless steel, b) standard CoCr alloy or standard MP35N alloy or a standard Phynox alloy or standard Elgiloy alloy or standard L605 alloy, c) standard TiAlV alloy, d) standard aluminum alloy, c) standard nickel alloy, f) standard titanium alloy, g) standard tungsten alloy, h) standard molybdenum alloy, i) standard copper alloy, j) standard beryllium-copper alloy, k) standard Nitinol alloy, 1) refractory metal alloy, or m) metal alloy that includes at least 5 atomic weight percent (awt. %) or atomic percent (awt %) rhenium (e.g., 5-99 awt. % rhenium and all values and ranges therebetween). In one non-limiting configuration, 10-100 wt. % of the frame includes refractory metal alloy, or a metal alloy that includes at least 15 atomic weight percent (awt. %) rhenium.

The inner skirt 300 can be formed of a variety of flexible materials (e.g., polymer (e.g., polyethylene terephthalate (PET), polyester, nylon, Kevlar, silicon, etc.), composite material, metal, fabric material, etc. In one non-limiting embodiment, the material used to partially or fully form the inner skirt 300 can be substantially non-clastic (i.e., substantially non-stretchable and non-compressible). In another non-limiting embodiment, the material used to partially or fully form the inner skirt 300 can be a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). The inner skirt 300 can optionally be formed from a combination of a cloth or fabric material that is coated with a flexible material or with a stretchable and/or compressible material so as to provide additional structural integrity to the inner skirt 300. The size, configuration and thickness of the inner skirt 300 is non-limiting (e.g., thickness of 0.1-20 mils and all values and ranges therebetween). The inner skirt 300 can be secured to the inside and/or outside of the frame 110 using various means (e.g., sutures, clips, clamp arrangement, etc.).

The inner skirt 300 can be used to 1) at least partially seal and/or prevent perivalvular leakage, 2) at least partially secure the leaflet structure 200 to the frame 110, 3) at least partially protect one or more of the leaflets of the leaflet structure 200 from damage during the crimping process of the prosthetic heat valve 100, 4) at least partially protect one or more of the leaflets of the leaflet structure 200 form damage during the operation of the prosthetic heart valve 100 in the heart H.

The prosthetic heart valve 100 can optionally include an outer skirt or sleeve (not shown) that is positioned at least partially about the exterior region of the frame 110. The outer skirt or sleeve, when used, generally is positioned completely around a portion of the outside of the frame 110. Generally, the outer skirt is positioned about the lower portion of the frame 110 and does not fully cover the upper portion of the frame 110; however, this is not required. The outer skirt can be connected to the frame 110 by a variety of arrangements (e.g., sutures, adhesive, melted connection, clamping arrangement, etc.). At least a portion of the outer skirt can optionally be located on the interior surface of the frame 110; however, this is not required. Generally, the outer skirt is formed of a more flexible and/or compressible material than the inner skirt 300; however, this is not required. The outer skirt can be formed of a variety of a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). The outer skirt can optionally be formed from a combination of a cloth or fabric material that is coated with the stretchable and/or compressible material so as to provide additional structural integrity to the outer skirt. The size, configuration and thickness of the outer skirt is non-limiting. The thickness of the outer skirt is generally 0.1-20 mils (and all values and ranges therebetween).

The leaflet structure 200 can be can be attached to the frame 110 and/or inner skirt 300. The connection arrangement used to secure the leaflet structure 200 to the frame 110 and/or inner skirt 300 is non-limiting (e.g., sutures, melted bold, adhesive, clamp arrangement, etc.). The material used to form the one or more leaflets of the leaflet structure 200 include, but are not limited to, bovine pericardial tissue, biocompatible synthetic materials, or various other suitable natural or synthetic materials.

The leaflet structure 200 can be comprised of two or more leaflets (e.g., 2, 3, 4, 5, 6, etc.). In one non-limiting arrangement, the leaflet structure 200 includes three leaflets that are arranged to collapse in a tricuspid arrangement. The size, shape and configuration of the one or more leaflets of the leaflet structure 200 are non-limiting. In one non-limiting arrangement, the leaflets have generally the same shape, size, configuration and thickness.

Two of more of the leaflets of the leaflet structure 200 can optionally be secured to one another at their adjacent sides to form commissures of the leaflet structure 200 (the edges where the leaflets come together). The leaflet structure 200 can be secured to the frame 110 and/or inner skirt 300 by a variety of connection arrangement (e.g., sutures, adhesive, melted bond, clamping arrangement, etc.).

One or more leaflets of the leaflet structure 200 can optionally include reinforcing structures or strips to 1) facilitate in securing the leaflets together, 2) facilitate in securing the leaflets to the inner skirt 300 and/or frame 110, and/or 3) inhibit or prevent tearing or other types of damage to the leaflets.

The prosthetic heart valve 100 is configured to be radially collapsible to a collapsed or crimped state for introduction into the body on a delivery catheter (FIG. 1B) and radially expandable to an expanded state for implanting the prosthetic heart valve 100 at a desired location in the heart (e.g., the aortic valve, etc.). The frame 110 of the prosthetic heart valve 100 is made of a plastically-expandable material (e.g., refractory metal alloy) that permits crimping of the frame 110 to a smaller profile for delivery and expansion of the prosthetic heart valve 100 using an expansion device such as the balloon B of a balloon catheter C.

Referring now to FIG. 1B, the prosthetic heart valve 100 is crimped into a portion of balloon B of the balloon catheter C. Various type of crimping apparatus and techniques can be used to crimp the prosthetic heart valve on the balloon delivery catheter. The process of crimping a prosthetic heart valve 100 using a crimping device is known in the art and will not be described herein. During a crimping procedure, damage to the leaflets of leaflet structure 200 should be avoided.

As illustrated in FIGS. 1C-1E, once prosthetic heart valve 100 is crimped on balloon B of a balloon delivery catheter C, balloon delivery catheter C is inserted through a blood vessel and to the location in heart H wherein prosthetic heart valve 100 is to be deployed (See FIG. 1C). At the treatment location, the balloon B on balloon delivery catheter C is expanded to thereby cause prosthetic heart valve 100 to be expanded and secured in a valve region A of heart H (See FIG. 1D). Thereafter, balloon B is deflated and balloon delivery catheter C is removed from the patient (See FIG. 1E).

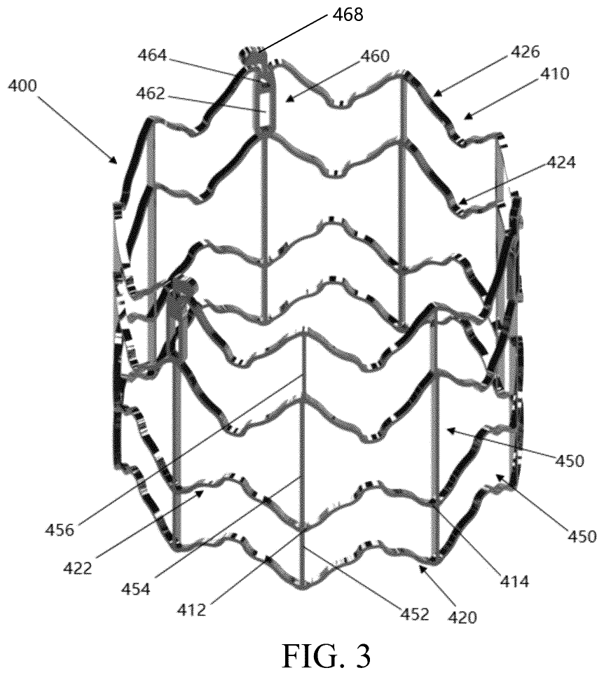

Referring now to FIGS. 3-5, other non-limiting configurations of frame 400 for prosthetic heart valve 100 are illustrated. The radially collapsible and expandable frame 400 includes plurality of angularly spaced struts 410, a plurality of axial struts 450, and a plurality of frame opening arrangements 460, and wherein angularly spaced struts 410, the plurality of axial struts 450, and frame opening arrangements 460 are connected together to form a plurality of cells 480 in frame 400. In one non-limiting configuration, the plurality of axial struts 450 are configured to limit the amount of foreshortening of the frame 400 (i.e., reduction in a longitudinal length of the frame when the frame is expanded from a crimped state to an expanded state) when the frame 400 of the prosthetic heart valve 100 is expanded. In one non-limiting specific configuration, the plurality of axial struts 450 are configured to limit the amount of foreshortening of the frame 400 to 0-20% (and all values and ranges therebetween) when the frame 400 of the prosthetic heart valve 100 is expanded.

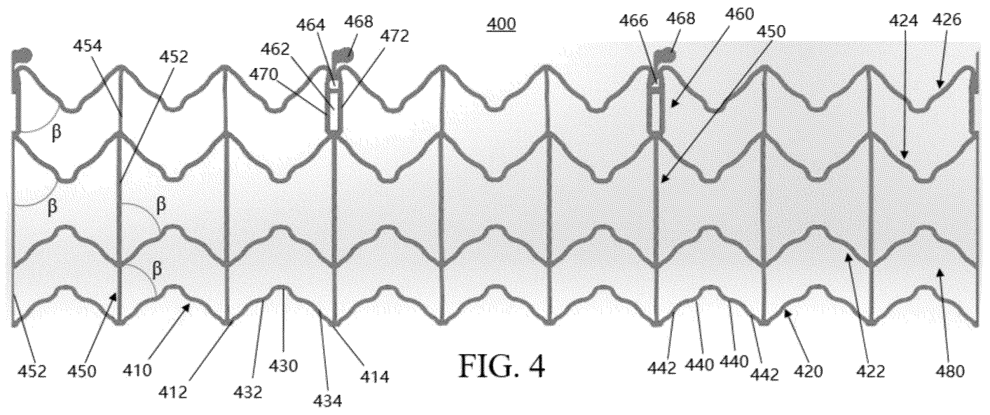

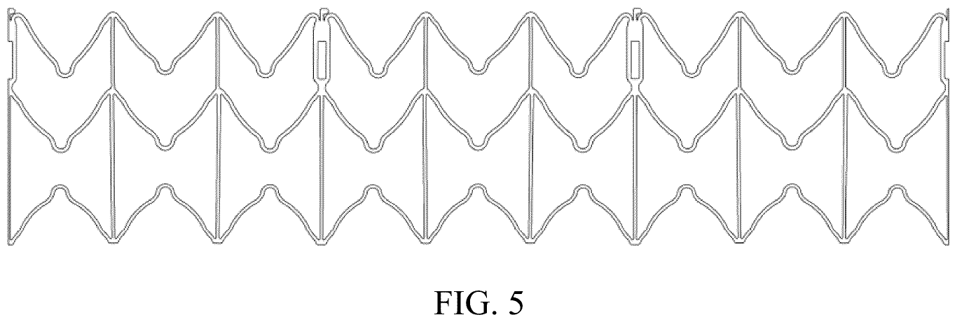

Angularly spaced struts 410 have first and second ends 412, 414, and angularly spaced struts 410 have first and second ends 412, 414 that are connected to axial struts 450 or frame opening arrangements 460. Frame opening arrangements 460 are located on the top portion of frame 400. Each of frame opening arrangements 460 can include a lower frame opening 462 and an optional an upper frame opening 464, 466. As illustrated in FIGS. 3 and 4, frame 400 is formed of three sets of cells, wherein each set of cells includes nine cells 480. As illustrated in FIG. 5, frame 400 includes three sets of cells, and wherein each set of cells includes six cells 480. As illustrated in FIGS. 3-5, the number, shape, and size of cells 480 in each of the three sets of cells are mirror images of one another, and have the same shape and size.

Referring again to FIGS. 3 and 4, a plurality of axial struts 450 that are formed of a three axial strut segments, 452, 454, 456, and some of axial struts 450 are formed of two axial strut segments. Frame 400 illustrated in FIG. 5 includes a plurality of axial struts 450 wherein some of the axial struts are formed of two axial strut segments and some of the axial struts are formed of a single axial strut segment. The thickness or cross-sectional area of each of axial struts 450 along the longitudinal axis of the axial strut can be constant or vary. The lower axial strut segments 452 can have a greater thickness or cross-sectional area than the upper axial strut segments 456. The middle axial strut segments 454 can have a greater thickness or cross-sectional area than upper axial strut segments 456. The lower axial strut segments 452 can have generally the same thickness or cross-sectional area as middle axial strut segments 454. As can be appreciated, lower axial strut segments 452 can have a different thickness or cross-sectional area as middle axial strut segments 454. The cross-sectional shape of each the axial struts 450 along the longitudinal length of axial strut 450 can be constant or vary. The longitudinal length of the axial strut segments can be the same or different. As illustrated in FIGS. 3 and 4, frame 400 includes a first row 420 of angularly spaced struts 410, a second row 422 of angularly spaced struts 410, a third row 424 of angularly spaced struts 410, and a fourth row 426 of angularly spaced struts 410.

Referring again to FIGS. 3-5, each of the angularly spaced struts 410 can be formed of a centrally located arcuate portion or semi-circular portion 430, and first and second arms 432, 434 that extend from each side of semi-circular portion 430. Each of first and second arms 432, 434 include one or more undulations 440, 442.

Referring now to FIGS. 3-5, frame opening arrangements 460 are located between third and fourth rows 424, 426 of angularly spaced struts 410. As can be appreciated, one or more frame opening arrangements 460 can be located on other regions of frame 400. Frame opening arrangements 460 can optionally be used as securing locations for one of more leaflet structures 200; however, it can be appreciated that one or more of frame opening arrangements 460 can optionally be used as securing locations for other structures (e.g., leaflet, inner skirt, outer skirt, etc.), and/or be used as an indicator of the orientation and/or location of frame 400 in a body passageway or heart valve. As illustrated in FIGS. 3-5, each of frame opening arrangements 460 includes first and second frame opening struts 470, 472 that form a lower frame opening 462 and an optional an upper frame opening 464, 466 therebetween. Referring now to FIGS. 3-5, frame opening arrangements 460 can optionally include one or more optional upper frame openings 464, 466. The one or more optional upper frame openings 464, 466 are generally positioned above lower frame opening 462. The top portion of each of frame opening arrangements 460 can optionally include a top marker 468. The shape and size of top marker 468 (when used) is non-limiting. The one or more top markers 468 (when used) can also or alternatively be used to enable one or more components of prosthetic heart valve 100 (e.g., leaflet, inner skirt, outer skirt, etc.) to be connected to frame 400. The one or more top markers 468 (when used) can also be used to facilitate in proper alignment and orientation of the prosthetic heart valve 100 in the heart valve prior to and/or during expansion of the prosthetic heart valve 100 in the heart valve.

The frame 110 of the prosthetic heart valve 100 can be configured such that it can be crimped onto a delivery catheter C so that the crimped prosthetic heart valve 100 can be inserted in heart valves of various sizes (e.g., less than 22 Fr; 24-27 FR (8-9 mm); etc.).

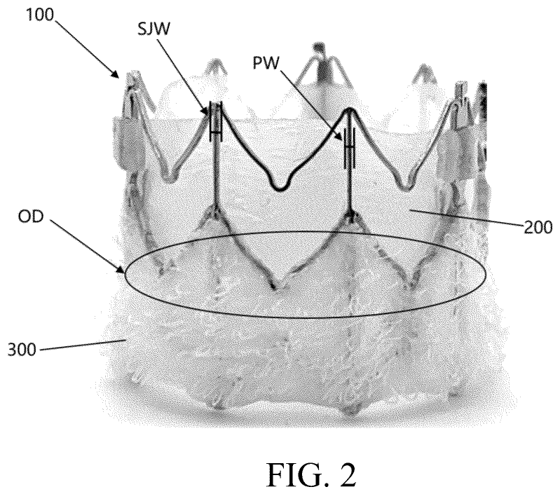

Referring now to FIG. 2, when the frame 110 is formed of a refractory metal alloy or metal alloy that includes at least 5 awt. % (e.g., 5-99 awt. % and all values and ranges therebetween) rhenium, the post width PW and/or the strut joint width SJW of a frame 110 that is formed of such metal alloy can be smaller than the post width PW and/or the strut joint width SJW of a similar shaped and configured frame formed of stainless steel, nitinol, Co—Cr alloy or TiAlV alloy, and still have the same or greater radial strength when the frame is expanded as compared to a frame formed of stainless steel, nitinol, Co—Cr alloy or TiAlV alloy.

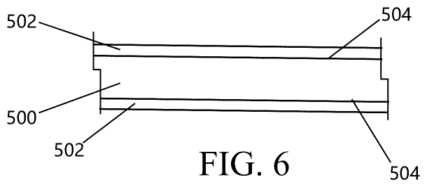

Referring now to FIG. 6, there is illustrated a cross-sectional view of a cross-section section 500 of a frame 110, 400 that illustrates an enhancement coating 502 on the outer surface 504 of the section of the frame. Although FIG. 6 only illustrated a coating on the outer surface of frame 110, 400, the enhancement coating 502 can also or alternatively be coated on one or more other components of the prosthetic heart valve 100 such as, but not limited to, the inner skirt, the outer skirt, one or more of all of the leaflets, and/or the material used to secure leaflets to frame. In one non-limiting configuration, 10-100% (and all values and ranges therebetween) of the outer surface 504 of frame 110, 400 is coated with enhancement coating 502. In another non-limiting configuration, the frame and one or more of the inner skirt, the outer skirt, and/or one or more of all of the leaflets are coated with enhancement coating 502, and 10-100% (and all values and ranges therebetween) of the outer surface 504 of frame 110, 400 is coated with enhancement coating 502, and 10-100% (and all values and ranges therebetween) of the outer surface of one or more of the inner skirt 300, the outer skirt, and/or one or more of all of the leaflets 200 are coated with enhancement coating 502.

The enhancement coating 502 can be used to improve one or more properties of the prosthetic heat valve (e.g., change exterior color of material having coated surface, increase surface hardness by use of the coated surface, increase surface toughness material having coated surface, reduced friction via use of the coated surface, improve scratch resistance of material that has the coated surface, improve impact wear of coated surface, improve resistance to corrosion and oxidation of coated material, form a non-stick coated surface, improve biocompatibility of material having the coated surface, reduce toxicity of material having the coated surface, reduce ion release from material having the coated surface, the enhancement coating forms a surface that is less of an irritant to cell about the coated surface after the prosthetic heart valve is implanted, reduces the rate to which cells grown on coated surface after prosthetic heart valve is implanted, reduce rate to which leaflets fail to properly operate after prosthetic heart valve is implanted, facilitate in nitric oxide generation on the surface of the coating, etc.).

Non-limiting enhancement coatings 502 that can be applied to a portion or all of the outer surface of one or more components of the prosthetic heart valve includes chromium nitride (CrN), diamond-like carbon (DLC), titanium nitride (TiN), titanium oxynitride or titanium nitride oxide (TiNOx), zirconium nitride (ZrN), zirconium oxide (ZrO₂), zirconium-nitrogen-carbon (ZrNC), zirconium OxyCarbide (ZrOC), zirconium oxynitride (ZrNxOy), and combinations of such coatings. In one one-limiting configuration, a portion or all of the outer surface of one or more components of the prosthetic heart valve includes titanium oxynitride or titanium nitride oxide (TiNOx) and/or zirconium oxynitride (ZrNxOy). The enhancement coating 502 can optionally be applied to a portion or all of the outer surface of one or more components of the prosthetic heart valve by a physical vapor deposition (PVD) process (e.g., sputter deposition, cathodic arc deposition or electron beam heating, etc.), chemical vapor deposition (CVD) process, atomic layer deposition (ALD) process, or a plasma-enhanced chemical vapor deposition (PE-CVD) process.

In one non-limiting embodiment, when forming a titanium oxynitride or titanium nitride oxide (TiNOx) coating on the prosthetic heart valve, the portion of the prosthetic heart valve that is to be coated can be optionally initially coated with Ti metal. The Ti metal coating, when applied, can be applied by PVD, CVD, ALD and PE-CVD in an inert environment. The coating thickness of Ti metal is 0.05-1 microns. Thereafter, the Ti metal coating is exposed to a nitrogen and oxygen mixture that can include nitrogen gas, oxygen gas, a nitrogen containing gas compound and/or an oxygen containing gas compound to cause the nitrogen and oxygen to react with the Ti metal coating. During the formation of the titanium oxynitride or titanium nitride oxide (TiNOx) coating, titanium particles can also be applied to the outer surface of the Ti metal coating prior to and/or during the exposure of the Ti metal coating to the nitrogen and oxygen mixture. The ratio of the N to the O can be varied to control the about of O in the TiNOx coating. The ratio of N to O when forming the TiNOx coating is generally 1:10 to 10:1 (and all values and ranges therebetween). The coating thickness of the TiNOx coating is generally 0.1-2 microns (and all values and ranges therebetween).

In another non-limiting embodiment, when forming a titanium oxynitride or titanium nitride oxide (TiNOx) coating on the prosthetic heart valve, the portion of the prosthetic heart valve that is to be coated is exposed to titanium particles and a nitrogen and oxygen mixture that can include nitrogen gas, oxygen gas, a nitrogen containing gas compound and/or an oxygen containing gas compound to cause the nitrogen and oxygen to react with the Ti particles. In this coating method, a Ti coating is not preapplied to the outer surface of any portion of the prosthetic heart valve that is to be coated with titanium oxynitride or titanium nitride oxide (TiNOx). The ratio of the N to the O can be varied to control the about of O in the TiNOx coating. The ratio of N to O when forming the TiNOx coating is generally 1:10 to 10:1 (and all values and ranges therebetween). The coating thickness of the TiNOx coating is generally 0.1-2 microns (and all values and ranges therebetween).

In one non-limiting embodiment, when forming a zirconium oxynitride (ZrNxOy) coating on the prosthetic heart valve, the portion of the prosthetic heart valve that is to be coated can be optionally initially coated with Zr metal. The Zr metal coating, when applied, can be applied by PVD, CVD, ALD and PE-CVD in an inert environment. The coating thickness of Zr metal is 0.05-1 microns. Thereafter, the Zr metal coating is exposed to a nitrogen and oxygen mixture that can include nitrogen gas, oxygen gas, a nitrogen containing gas compound and/or an oxygen containing gas compound to cause the nitrogen and oxygen to react with the Zr metal coating. During the formation of the zirconium oxynitride (ZrNxOy) coating, zirconium particles can also be applied to the outer surface of the Zr metal coating prior to and/or during the exposure of the Zr metal coating to the nitrogen and oxygen mixture. The ratio of the N to the O can be varied to control the about of O in the ZrNxOy coating. The ratio of N to O when forming the ZrNxOy coating is generally 1:10 to 10:1 (and all values and ranges therebetween). The coating thickness of the ZrNxOy coating is generally 0.1-2 microns (and all values and ranges therebetween).

In another non-limiting embodiment, when forming a zirconium oxynitride (ZrNxOy) coating on the prosthetic heart valve, the portion of the prosthetic heart valve that is to be coated is exposed to zirconium particles and a nitrogen and oxygen mixture that can include nitrogen gas, oxygen gas, a nitrogen containing gas compound and/or an oxygen containing gas compound to cause the nitrogen and oxygen to react with the Zr particles. In this coating method, a Zr coating is not preapplied to the outer surface of any portion of the prosthetic heart valve that is to be coated with zirconium oxynitride (ZrNxOy) coating. The ratio of the N to the O can be varied to control the about of O in the ZrNxOy coating. The ratio of N to O when forming the ZrNxOy coating is generally 1:10 to 10:1 (and all values and ranges therebetween). The coating thickness of the ZrNxOy coating is generally 0.1-2 microns (and all values and ranges therebetween).

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall therebetween.

To aid the Patent Office and any readers of this application and any resulting patent in interpreting the claims appended hereto, applicants do not intend any of the appended claims or claim elements to invoke 35 U.S.C. 112 (f) unless the words “means for” or “step for” are explicitly used in the particular claim.

1. A valve that that is configured to be implanted in a patient; said valve includes a plurality of components; two of said components include a metallic frame and at least one leaflet; said metallic frame is directly or indirectly attached to said at least one leaflet; said at least one leaflet is configured to at least partially control fluid flow through said metallic frame; a portion or all of an outer surface of at least one component of said valve includes a layer of an enhancement material; said enhancement material is formulated to i) provide nitric oxide after said valve is implanted in the patient, and/or ii) promote generation of nitric oxide after said valve is implanted in the patient; said enhancement material is at least partially formulated of oxynitride.

2. The prosthetic heart valve as defined in claim 1, wherein said enhancement material includes a metal oxynitride layer.

3. The prosthetic heart valve as defined in claim 2, wherein said metal oxynitride includes titanium oxynitride and/or zirconium oxynitride.

4. The prosthetic heart valve as defined in claim 2, wherein said metal oxynitride layer has a thickness of at least 10 nanometers.

5. The prosthetic heart valve as defined in claim 2, wherein said metal oxynitride has an oxygen to nitrogen atomic ratio of 1:10 to 10:1.

6. The prosthetic heart valve as defined in claim 2, wherein said layer of enhancement material includes a metallic adhesion layer; said metal oxynitride is at least partially coated on an outer surface of said metallic adhesion layer and said metallic adhesion layer is coated on an outer surface of said metallic frame.

7. The prosthetic heart valve as defined in claim 6, wherein said metallic adhesion layer includes titanium metal or zirconium metal.

8. The prosthetic heart valve as defined in claim 6, wherein said metallic adhesion layer has a thickness of at least 1 nanometers.

9. The prosthetic heart valve as defined in claim 1, wherein said plurality of components further include one or more of an inner skirt, an outer skirt, and/or sutures.

10. The prosthetic heart valve as defined in claim 9, wherein said plurality of components include said frame, said at least one leaflet, said inner skirt, said outer skirt, and said sutures.

11. The prosthetic heart valve as defined in claim 1, wherein said layer of enhancement material includes no more than 0.1 wt. % nickel and/or no more than 0.1 wt. % cobalt.

12. The prosthetic heart valve as defined in claim 1, wherein said metallic frame includes no more than 0.1 wt. % nickel and/or no more than 0.1 wt. % cobalt.

13. The prosthetic heart valve as defined in claim 1, wherein said layer of enhancement material is only coated on or over an outer surface of at least a portion of said metallic frame.

14. The prosthetic heart valve as defined in claim 1, wherein said layer of enhancement is coated on or over at least a portion of an outer surface of said metallic frame and on or over an outer surface of said at least one leaflet.

15. The prosthetic heart valve as defined in claim 9, wherein said layer of enhancement is coated on or over at least a portion of a) an outer surface of said metallic frame, b) an outer surface of said at least one leaflet, c) an outer surface of said inner skirt and d) an outer surface of said outer skirt.

16. The prosthetic heart valve as defined in claim 1, wherein said metallic frame is configured to foreshorten 0-5% of a longitudinal length of said metallic frame when said metallic frame is expanded from a crimped state to an expanded state.

17. The prosthetic heart valve as defined in claim 1, wherein said metallic frame is formed of a) standard stainless steel, b) standard cobalt-chromium alloy, c) standard titanium-aluminum-vanadium alloy, d) standard aluminum alloy, e) standard nickel alloy, f) standard titanium alloy, g) standard tungsten alloy, h) standard molybdenum alloy, i) standard copper alloy, j) standard beryllium-copper alloy, k) standard titanium-nickel alloy, 1) refractory metal alloy, or m) metal alloy that includes at least 5 atomic weight percent (awt. %) rhenium.

18. The prosthetic heart valve as defined in claim 17, wherein said metallic frame is formed of said refractory metal alloy or said metal alloy that includes at least 15 atomic weight percent (awt. %) rhenium.

19. A method for repairing a valve; said method comprising:
a. providing a valve that is crimped about a delivery system; said valve includes a plurality of components; two of said components include a metallic frame and at least one leaflet; said metallic frame is directly or indirectly attached to said at least one leaflet; said at least one leaflet is configured to at least partially control fluid flow through said metallic frame; a portion or all of an outer surface of at least one component of said valve includes a layer of enhancement material; said enhancement material is formulated to i) provide nitric oxide after said valve is implanted in the patient, and/or ii) promote generation of nitric oxide; said enhancement material is at least partially formulated of oxynitride after said valve is implanted in the patient;
b. positioning said valve in a treatment area in a patient; and,
c. expanding said metallic frame from a crimped state to an expanded state while said prosthetic heart valve is in said treatment area of said heart.

20. The method as defined in claim 19, wherein said metallic frame has no more than 5% recoil after said metallic frame has been expanded from said crimped state to said expanded state.

21. The method as defined in claim 19, wherein said enhancement material includes metal oxynitride.

22. The method as defined in claim 21, wherein said metal oxynitride includes titanium oxynitride and/or zirconium oxynitride.

23. The method as defined in claim 21, wherein said metal oxynitride has a thickness of at least 10 nanometers.

24. The method as defined in claim 21, wherein said metal oxynitride has an oxygen to nitrogen atomic ratio of 1:10 to 10:1.

25. The method as defined in claim 21, wherein said layer of enhancement material includes a metallic adhesion layer; said metal oxynitride is at least partially coated on an outer surface of said metallic adhesion layer and said metallic adhesion layer is coated on an outer surface of said metallic frame.

26. The method as defined in claim 25, wherein said metallic adhesion layer includes titanium metal or zirconium metal.

27. The method as defined in claim 25, wherein said metallic adhesion layer has a thickness of at least 1 nanometers.

28. The method as defined in claim 19, wherein said plurality of components further include one or more of an inner skirt, an outer skirt, and/or sutures.

29. The method as defined in claim 28, wherein said plurality of components include said frame, said at least one leaflet, said inner skirt, said outer skirt, and said sutures.

30. The method as defined in claim 19, wherein said layer of enhancement material includes no more than 0.1 wt. % nickel and/or no more than 0.1 wt. % cobalt.

31. The method as defined in claim 19, wherein said metallic frame includes no more than 0.1 wt. % nickel and/or no more than 0.1 wt. % cobalt.

32. The method as defined in claim 19, wherein said layer of enhancement material is only coated on or over an outer surface of at least a portion of said metallic frame.

33. The method as defined in claim 19, wherein said layer of enhancement is coated on or over at least a portion of an outer surface of said metallic frame and on or over an outer surface of said at least one leaflet.

34. The method as defined in claim 28, wherein said layer of enhancement is coated on or over at least a portion of a) an outer surface of said metallic frame, b) an outer surface of said at least one leaflet, c) an outer surface of said inner skirt and d) an outer surface of said outer skirt.

35. The method as defined in claim 19, wherein said metallic frame is configured to foreshorten 0-5% of a longitudinal length of said metallic frame when said metallic frame is expanded from a crimped state to an expanded state.

36. The method as defined in claim 19, wherein said metallic frame is formed of a) standard stainless steel, b) standard cobalt-chromium alloy, c) standard titanium-aluminum-vanadium alloy, d) standard aluminum alloy, e) standard nickel alloy, f) standard titanium alloy, g) standard tungsten alloy, h) standard molybdenum alloy, i) standard copper alloy, j) standard beryllium-copper alloy, k) standard titanium-nickel alloy, l) refractory metal alloy, or m) metal alloy that includes at least 5 atomic weight percent (awt. %) rhenium.

37. The method as defined in claim 36, wherein said metallic frame is formed of said refractory metal alloy or said metal alloy that includes at least 15 atomic weight percent (awt. %) rhenium.

38. The method as defined in claim 19, wherein said nitric oxide donation includes use of a nitric oxide donating compound; said nitric oxide donating compound is a) a direct nitric oxide donator, wherein said direct nitric oxide donator includes S—NO—N-acetyl-L-cysteine, Molsidomine, Diethylamino-NONOate, Spermine NONOate, S—NO-Glutathione, and/or S—NO-diclofenac, b) a metabolic nitric oxide donator, wherein said metabolic nitric oxide donator includes nitroglycerin, amyl nitrite, isosorbide dinitrate, isosorbide mononitrate, and/or nicorandil, and/or c) a bifunctional nitric oxide donator, wherein said bifunctional nitric oxide donator includes nitroaspirins and/or S-Nitroso-NSAIDs.

39. The method as defined in claim 38, wherein at least one of said leaflets is formed of a biological tissue material, and wherein said nitric oxide donating compound is a) adhered to and/or permeated within interstices of said biological tissue material, b) chemically bound to an extracellular matrix of said biological tissue material, and/or c) chemically bound to free amine residues on collagen of said biological tissue material via crosslinking.

40. The method as defined in claim 39, wherein said crosslinking is at least partially achieved by use of one or more of glutaraldehyde, formaldehyde, genipin, carbodiimides, dialdehyde starch, temperature, and/or UV light crosslinking.

41. The method as defined in claim 39, wherein said crosslinking is reduced via a reducing agent to inhibit or prevent reversibility of said cross-linking.

42. The method as defined in claim 19, wherein said prosthetic heart valve includes biological tissue material with a nitric oxide donating compound that is chemically bound to a secondary structure acting as an intermediary between said nitric oxide donating compound and collagen and/or said nitric oxide donating compound and a crosslinking agent.

43. The method as defined in claim 42, wherein said secondary structure possesses residues congruent with crosslinking of tissue-based collagen structures; said residues include aldehyde residues, carboxyl residues, and/or amine residues.

44. The method as defined in claim 42, wherein said nitric oxide donating compound is embedded within said interstices of said biological tissue material.

45. The method as defined in claim 44, wherein said embedded nitric oxide donating compound that is retained within said biological tissue material is configured to release nitric oxide into said local environment.

46. The method as defined in claim 44, wherein said embedded nitric oxide donating compound itself is released into said local environment.

47. The method as defined in claim 44, wherein said nitric oxide donating compound is introduced into interstices of said biological tissue material via serial immersion into a treatment solution then drying said treated biological tissue material.

48. The method as defined in claim 47, wherein said treatment solution includes said nitric oxide donor compound that is within a dimensional stabilizer compound that enables said treated biological tissue material be stable in standard air composition.

49. The method as defined in claim 48, wherein said dimensional stabilizer compound includes a polyol compound.

50. The method as defined in claim 49, wherein said polyol compound includes ethylene glycol, propylene glycol, and/or glycerol.

51. The method as defined in claim 42, wherein said biological tissue material is treated with a dimensional stabilizer compound at a time that is concurrent with or subsequent to treatment adherence of said nitric oxide donor compound.

52. The method as defined in claim 42, wherein said secondary structures includes a polymeric material with nitric oxide generating compound that is adhered to or is permeated within pores of said polymeric material.

53. The method as defined in claim 52, wherein said polymetric material includes polyethers, polyesters, polyurethanes, and/or polycarbons.

54. The method as defined in claim 52, wherein said polymetric material includes one or more compositional elements; said compositional elements includes macrodiol segments, polyol segments, and/or cyanates.

55. A valve that that is configured to be implanted in a patient; said valve includes an expandable metallic frame and a plurality of leaflets; said expandable metallic frame is directly or indirectly attached to said plurality of leaflets; said plurality of leaflet is configured to at least partially control fluid flow through said expandable metallic frame when said valve is implanted in a patient; said metallic frame is configured to foreshorten 0-5% of a longitudinal length of said metallic frame when said metallic frame is expanded from a crimped state to an expanded state; said expandable metallic frame is formed of a) standard stainless steel, b) standard cobalt-chromium alloy, c) standard titanium-aluminum-vanadium alloy, d) standard aluminum alloy, e) standard nickel alloy, f) standard titanium alloy, g) standard tungsten alloy, h) standard molybdenum alloy, i) standard copper alloy, j) standard beryllium-copper alloy, k) standard titanium-nickel alloy, 1) refractory metal alloy, or m) metal alloy that includes at least 5 atomic weight percent (awt. %) rhenium; a portion or all of an outer surface of said expandable metallic frame and/or said plurality of leaflets includes a layer of an enhancement material; said layer of enhancement material includes no more than 0.1 wt. % nickel and/or no more than 0.1 wt. % cobalt; said enhancement material is formulated to i) provide nitric oxide after said valve is implanted in the patient, and/or ii) promote generation of nitric oxide after said valve is implanted in the patient; said enhancement material is at least partially formulated of metal oxynitride; said metal oxynitride includes titanium oxynitride and/or zirconium oxynitride; a thickness of said coating of said enhancement material is less than a thickness of said expandable metallic frame and is less than a thickness of each of said leaflets; said coating of said enhancement materials has a thickness of at least 10 nanometers; said layer of enhancement coating material is a) a coating of said metal oxynitride or b) a coating of a metallic adhesion layer and a coating of said metal oxynitride on a surface of said metallic adhesion layer, and wherein said metallic adhesion layer includes titanium metal or zirconium metal; said layer of enhancement material is directly coated on an outer surface of said expandable metallic frame and/or an outer surface of one or more of said plurality of leaflets.

56. The prosthetic heart valve as defined in claim 55, said layer of enhancement material is only coated on or over an outer surface of at least a portion of said expandable metallic frame.

57. The prosthetic heart valve as defined in claim 55, wherein said layer of enhancement is coated on or over at least a portion of an outer surface of said metallic frame and on or over an outer surface of one or more of said plurality of leaflets.

58. The prosthetic heart valve as defined in claim 55, wherein said metallic frame is formed of said refractory metal alloy or said metal alloy that includes at least 15 atomic weight percent (awt. %) rhenium.

59. The prosthetic heart valve as defined in claim 56, wherein said metallic frame is formed of said refractory metal alloy or said metal alloy that includes at least 15 atomic weight percent (awt. %) rhenium.

60. The prosthetic heart valve as defined in claim 57, wherein said metallic frame is formed of said refractory metal alloy or said metal alloy that includes at least 15 atomic weight percent (awt. %) rhenium.
